(19) United States
(12) Patent Application Publication
(10) Pub. No.: US 2008/0026366 A1
(43) Pub. Date: Jan. 31, 2008

(54) Biological fixative and method of using the biological fixative

(75) Inventor: Lualhati E. Harkins, Birmingham, AL (US)

Correspondence Address: LAW OFFICE OF PHILLIP F. FOX, 10985 40TH PLACE NORTH, PLYMOUTH, MN, 55441, US

(73) Assignee: Newcomer Supply, Inc., Middleton, WI (US)

(21) Appl. No.: 11/810,349

(22) Filed: Jun. 5, 2007

Related U.S. Application Data
(60) Provisional application No. 60/811,479, filed on Jun. 7, 2006.

(51) Int. Cl. C12Q 1/70; G01N 1/30; G01N 33/48

(52) U.S. Cl. 435/5; 435/40.5; 435/40.51; 435/40.52; 436/18

(57) ABSTRACT

A composition that includes an aldehyde, alcohol, and a ketone, the volumetric ratio of the alcohol to the ketone in the composition ranging from as low as about 0.8:1 to as high as about 4.5:1 and the volumetric ratio of the alcohol to the aldehyde in the composition ranging from as low as about 41.5:1 to as high as about 450:1.

### CROSS-REFERENCE TO RELATED APPLICATION(S)

This application claims the benefit of priority from U.S. Provisional Patent Application Ser. No. 60/811,479 entitled Biological Fixative And Method Of Using The Biological Fixative that was filed on Jun. 7, 2006 under any and all applicable U.S. statutes, including 35 U.S.C. §119(e). The entire content of U.S. Provisional Patent Application Ser. No. 60/811,479 entitled Biological Fixative And Method Of Using The Biological Fixative that was filed on Jun. 7, 2006 is incorporated by reference in this application.

### BACKGROUND OF THE INVENTION

The present invention generally relates to a biological fixative and to methods of using the biological fixative to stabilize (“fix”) biological samples. More specifically, the present invention relates to a biological fixative that may beneficially be employed to quickly and efficiently stabilize (“fix”) frozen and previously frozen biological samples and support rapid analytical and recovery processing.

Various biological fixatives are employed for purposes of attempting to stabilize biological materials for future analysis and study. Desirably, biological fixatives will stabilize the morphology—that is the structure of the biological material—to be as close to the structure of the biological material when the biological material was extracted from the living being, as possible. Also, biological fixatives will desirably stabilize the antigenicity of the biological material to be as close to the antigenicity of the biological material when the biological material was extracted from the living being, as possible. Many existing biological fixatives function better at stabilizing morphology at the expense of antigenicity or function better at stabilizing antigenicity at the expense of morphology. Further some existing biological fixatives really are not very good at stabilizing either morphology or antigenicity.

Speed of fixation is also a factor with biological fixatives. While some biological fixatives do a fair job of stabilizing morphology and antigenicity, this success tends to come at the expense of quickly completing fixation of the biological material. Intraoperative consultations where a patient remains in surgery and immediately available for further procedures, should the results of the intraoperative consultation so dictate, require the ability to rapidly fix the biological material obtained from the patient. Time-consuming fixation approaches that may obtain good morphology and antigenicity stabilization become obsolete when such rapid intraoperative consultations are required.

While existing biological fixatives and procedures have helped improve the knowledge base with regard to biological fixation, further advances are needed. Such advances will desirably enhance both morphology and antigenicity stabilization while allowing for simpler and more rapid fixative approaches. The biological fixative and fixation methods of the present invention have surprisingly been found to achieve both superior morphology and antigenicity stabilization while supporting simpler and rapid fixation of biological materials.

### BRIEF SUMMARY OF THE INVENTION

The present invention encompasses a composition that includes an aldehyde, alcohol, and a ketone. The volumetric ratio of the alcohol to the ketone in the composition may range from as low as about 0.8:1 to as high as about 4.5:1 and the volumetric ratio of the alcohol to the aldehyde in the composition may range from as low as about 41.5:1 to as high as about 450:1. The present invention further includes various materials and various methods.

### BRIEF DESCRIPTION OF THE DRAWINGS

FIG. 1 is a micrograph visually depicting immuno localization of the antibody FCR 5 in human tonsil tissue fixed using a biological fixative of the present invention in accordance with a fixation method of the present invention.

FIG. 1A is a micrograph visually depicting immuno localization of the antibody FCR 5 in human tonsil tissue fixed using a comparative fixation technique employing ethanol.

FIG. 1B is a micrograph visually depicting immuno localization of the antibody FCR 5 in human tonsil tissue fixed using a comparative fixation technique employing methanol.

FIG. 1C is a micrograph visually depicting immuno localization of the antibody FCR 5 in human tonsil tissue fixed using a comparative fixation technique employing formalin.

FIG. 1D is a micrograph visually depicting immuno localization of the antibody FCR 5 in human tonsil tissue fixed using a comparative fixation technique employing acetone.

FIG. 2 is a micrograph visually depicting immuno localization of the antibody FCR 1 in human tonsil tissue fixed using a biological fixative of the present invention in accordance with a fixation method of the present invention.

FIG. 2A is a micrograph visually depicting immuno localization of the antibody FCR 1 in human tonsil tissue fixed using a comparative fixation technique employing formalin.

FIG. 2B is a micrograph visually depicting immuno localization of the antibody FCR 1 in human tonsil tissue fixed using a comparative fixation technique employing formalin and also employing proteolytic enzyme (trypsin) pre-treatment.

FIG. 3 is a micrograph visually depicting immuno localization of the protein Actin in a human brain tumor fixed using a biological fixative of the present invention in accordance with a fixation method of the present invention.

FIG. 3A is a micrograph visually depicting immuno localization of the protein Actin in a human brain tumor fixed using a comparative fixation technique employing ethanol.

FIG. 3B is a micrograph visually depicting immuno localization of the protein Actin in a human brain tumor fixed using a comparative fixation technique employing methanol.

FIG. 3C is a micrograph visually depicting immuno localization of the protein Actin in a human brain tumor fixed using a comparative fixation technique employing formalin.

FIG. 3D is a micrograph visually depicting immuno localization of the protein Actin in a human brain tumor fixed using a comparative fixation technique employing acetone.

FIG. 4 is a micrograph visually depicting immuno localization of the protein Actin in normal human brain tissue fixed using a biological fixative of the present invention in accordance with a fixation method of the present invention.

FIG. 4A is a micrograph visually depicting immuno localization of the protein Actin in normal human brain tissue fixed using a comparative fixation technique employing methanol.

FIG. 4B is a micrograph visually depicting immuno localization of the protein Actin in normal human brain tissue fixed using a comparative fixation technique employing ethanol.

FIG. 4C is a micrograph visually depicting immuno localization of the protein Actin in normal human brain tissue fixed using a comparative fixation technique employing formalin.

FIG. 4D is a micrograph visually depicting immuno localization of the protein Actin in normal human brain tissue fixed using a comparative fixation technique employing formalin and also employing proteolytic enzyme (trypsin) pre-treatment.

FIG. 4E is a micrograph visually depicting immuno localization of the protein Actin in normal human brain tissue fixed using a comparative fixation technique employing acetone.

FIG. 5 is a micrograph visually depicting immuno localization of Cytomegalovirus in a human brain tumor fixed using a biological fixative of the present invention in accordance with a fixation method of the present invention.

FIG. 5A is a micrograph visually depicting immuno localization of Cytomegalovirus in a human brain tumor fixed using a comparative fixation technique employing formalin.

FIG. 5B is a micrograph visually depicting immuno localization of Cytomegalovirus in a human brain tumor fixed using a comparative fixation technique employing ethanol.

FIG. 5C is a micrograph visually depicting immuno localization of Cytomegalovirus in a human brain tumor fixed using a comparative fixation technique employing methanol.

FIG. 5D is a micrograph visually depicting immuno localization of Cytomegalovirus in a human brain tumor fixed using a comparative fixation technique employing acetone.

FIG. 6 is a micrograph visually depicting, via immunofluorescence, labeling of the MHC Class II antibody in murine epithelial tissue fixed using a biological fixative of the present invention in accordance with a fixation method of the present invention.

FIG. 6A is a micrograph visually depicting, via immunofluorescence, labeling of the MHC Class II antibody in murine epithelial tissue fixed using a comparative fixation technique employing ethanol.

FIG. 6B is a micrograph visually depicting, via immunofluorescence, labeling of the MHC Class II antibody in murine epithelial tissue fixed using a comparative fixation technique employing methanol.

FIG. 6C is a micrograph visually depicting, via immunofluorescence, labeling of the MHC Class II antibody in murine epithelial tissue fixed using a comparative fixation technique employing formalin.

FIG. 6D is a micrograph visually depicting, via immunofluorescence, labeling of the MHC Class II antibody in murine epithelial tissue fixed using a comparative fixation technique employing acetone.

FIG. 7 is a micrograph visually depicting, via immunofluorescence, labeling of the antibody CD11c in murine epithelial tissue fixed using a biological fixative of the present invention in accordance with a fixation method of the present invention.

FIG. 7A is a micrograph visually depicting, via immunofluorescence, labeling of the antibody CD11c in murine epithelial tissue fixed using a comparative fixation technique employing ethanol.

FIG. 7B is a micrograph visually depicting, via immunofluorescence, labeling of the antibody CD11c in murine epithelial tissue fixed using a comparative fixation technique employing methanol.

FIG. 7C is a micrograph visually depicting, via immunofluorescence, labeling of the antibody CD11c in murine epithelial tissue fixed using a comparative fixation technique employing formalin.

FIG. 7D is a micrograph visually depicting, via immunofluorescence, labeling of the antibody CD 11c in murine epithelial tissue fixed using a comparative fixation technique employing acetone.

FIG. 8 is a micrograph visually depicting, via immunofluorescence, labeling of the antibody NFK-β and labeling of Cytomegalovirus in Cytomegalovirus-infected tissue culture fixed using a biological fixative of the present invention in accordance with a fixation method of the present invention.

FIG. 8A is a micrograph visually depicting, via immunofluorescence, labeling of the antibody NFK-β and labeling of Cytomegalovirus in Cytomegalovirus-infected tissue culture fixed using a comparative fixation technique employing ethanol.

FIG. 8B is a micrograph visually depicting, via immunofluorescence, labeling of the antibody NFK-β and labeling of Cytomegalovirus in Cytomegalovirus-infected tissue culture fixed using a comparative fixation technique employing methanol.

FIG. 8C is a micrograph visually depicting, via immunofluorescence, labeling of the antibody NFK-β and labeling of Cytomegalovirus in Cytomegalovirus-infected tissue culture fixed using a comparative fixation technique employing acetone.

FIG. 8D is a micrograph visually depicting, via immunofluorescence, labeling of the antibody NFK-β and labeling of Cytomegalovirus in Cytomegalovirus-infected tissue culture fixed using a comparative fixation technique employing formalin.

FIG. 9 is a micrograph visually depicting, via immunofluorescence, labeling of the MHC Class II antibody in murine epithelial tissue fixed using a biological fixative of the present invention in accordance with a fixation method of the present invention.

FIG. 9A is a micrograph visually depicting, via immunofluorescence, labeling of the MHC Class II antibody in murine epithelial tissue fixed using a comparative fixation technique employing formalin.

FIG. 9B is a micrograph visually depicting, via immunofluorescence, labeling of the MHC Class II antibody in murine epithelial tissue fixed using a comparative fixation technique employing ethanol.

FIG. 9C is a micrograph visually depicting, via immunofluorescence, labeling of the MHC Class II antibody in murine epithelial tissue fixed using a comparative fixation technique employing methanol.

FIG. 9D is a micrograph visually depicting, via immunofluorescence, labeling of the MHC Class II antibody in murine epithelial tissue fixed using a comparative fixation technique employing acetone.

### DETAILED DESCRIPTION

The present invention generally relates to a biological fixative and to methods of using the biological fixative to stabilize and fix biological samples. More specifically, the present invention relates to a biological fixative that may beneficially be employed to quickly and efficiently stabilize frozen and previously frozen biological samples and support rapid analytical studies.

All percentages and ratios stated herein are on a volume basis, unless otherwise indicated. Unless otherwise indicated herein, room temperature means a temperature of about 22° C.

The biological fixative of the present invention may alternatively and equivalently be referred to as a biological stabilizer. The biological fixative, which may also be referred to as a “fixative composition,” includes several different components. For example, the biological fixative includes an aldehyde, alcohol, and a ketone, along with pH buffering components. Thus, the biological fixative is typically pH buffered. The aldehyde may be an alkanal, such as a C₁ to C₆ alkanal, including, for example, formaldehyde. The aldehyde may be incorporated into the biological fixative in any available form. For example, where the aldehyde is formaldehyde, it has been found convenient to incorporate the formaldehyde in the biological fixative by incorporating neutral buffered formalin (an aqueous solution) in the biological fixative. The formalin typically incorporates a small amount of methanol, since methanol is commonly employed as a solvent for facilitating aqueous solution of the normally gaseous formaldehyde in liquid water to form formalin. Besides the methanol component of the formalin, the biological fixative may also incorporate, other alcohols, such as alkanols, including, for example, ethanol. Some examples of suitable ketones include acetone and methyl ethyl ketone (sometimes referred to as “MEK”).

While alcohols, such as alkanols, are included in the biological fixative of the present invention, embodiments of the biological fixative are generally free, or essentially free, of polyols, including diols, triols, etc. Such polyols excluded, or essentially excluded, from the biological fixative include glycol (glycerol), polyethylene glycol, ethylene glycol, sorbitol, mannitol, and the like.

If formaldehyde is incorporated directly in the biological fixative as formaldehyde, rather than as part of formalin, the formaldehyde will typically be incorporated as a solution of formaldehyde in aqueous solution with the methanol. Commercially available aqueous formaldehyde solutions typically include about 37 to about 40 volume percent formaldehyde, about 11 to about 14 volume percent methanol, and sufficient water to make 100 volume percent. Ten volume percent formalin solutions are one commercially available form of formalin. As an example, a ten volume percent formalin solution may include about ten volume percent of a commercially available aqueous formaldehyde solution (containing about 37 to about 40 volume percent formaldehyde, see above) and water to make 100 volume percent. Commercially available formalin solutions typically are also pH buffered and therefore may include common pH buffering agents, such as sodium phosphate monobasic and sodium phosphate dibasic (anhydrous).

The pH of the biological fixative will ordinarily range from about 6 to about 7 standard pH units at room temperature of about 22° C. In one exemplary formulation, the pH of the biological fixative is about 7 standard pH units at room temperature of about 22° C. The pH of the biological fixative may be adjusted, particularly within the range from about 6 to about 7 standard pH units at room temperature, depending upon the biological sample being fixed, to optimize the results, such as staining or labeling characteristics, of the analysis procedure performed on the biological sample following fixation of the biological sample in accordance with the present invention.

Some exemplary formulation options for the biological fixative of the present invention are shown in Table 1, where all concentrations and quantities are understood to be modified by the word “about”:

TABLE 1

| | CONCENTRATION | |
|---|---|---|
| COMPONENT | OR QUANTITY | UNITS |
| Formaldehyde^{C} | 0.1 to 0.6 | Volume percent^{A} |
| Ketone | 10 to 30 | Volume percent^{A} |
| Ethanol^{D} | 25 to 45 | Volume Percent^{A} |
| Methanol^{C} | 0.03 to 0.22 | Volume Percent^{A} |
| Water | 25 to 65 | Volume Percent^{A} |
| Sodium Phosphate Monobasic | 0.05 to 0.34 | Grams/1000 ml^{B} |
| Sodium Phosphate Dibasic | 0.2 to 1.2 | Grams/1000 ml^{B} |
| (Anhydrous) | | |
| Sodium Chloride | 1.1 to 6.8 | Grams/1000 ml^{B} |

^{A}Based on the total volume of the formaldehyde, ketone, ethanol, methanol, and water
^{B}Based on the total volume of the formaldehyde, ketone, ethanol, methanol, and water being 1000 milliliters
^{C}Provided as part of an aqueous solution containing about 37 to 40 volume percent formaldehyde, about 11 to about 14 volume percent methanol, and the balance water
^{D}Provided as an aqueous solution containing about 95 volume percent, or more, ethanol and about 2 volume percent, or less, of a denaturant, such as methanol, and the balance water

Still more exemplary formulation options for the biological fixative of the present invention are shown in Table 2, where all concentrations and quantities are understood to be modified by the word “about”:

TABLE 2

| | CONCENTRATION | |
|---|---|---|
| COMPONENT | OR QUANTITY | UNITS |
| Formaldehyde^{C} | 0.25 to 0.35 | Volume percent^{A} |
| Ketone | 18 to 22 | Volume percent^{A} |
| Ethanol^{D} | 34 to 38 | Volume Percent^{A} |
| Methanol^{C} | 0.075 to 0.13 | Volume Percent^{A} |
| Water | 38 to 45 | Volume Percent^{A} |
| Sodium Phosphate Monobasic | 0.16 to 0.18 | Grams/1000 ml^{B} |
| Sodium Phosphate Dibasic | 0.58 to 0.6 | Grams/1000 ml^{B} |
| (Anhydrous) | | |
| Sodium Chloride | 3.2 to 3.6 | Grams/1000 ml^{B} |

^{A}Based on the total volume of the formaldehyde, ketone, ethanol, methanol, and water
^{B}Based on the total volume of the formaldehyde, ketone, ethanol, methanol, and water being 1000 milliliters
^{C}Provided as part of an aqueous solution containing about 37 to 40 volume percent formaldehyde, about 11 to about 14 volume percent methanol, and the balance water
^{D}Provided as an aqueous solution containing about 95 volume percent, or more, ethanol and about 2 volume percent, or less, of a denaturant, such as methanol, and the balance water

A biological fixative with a formulation falling within the parameters provided in Table 2 may be obtained as FROZFIX® biological fixative from Newcomer Supply of Middleton, Wis.

Some exemplary formulation options for the biological fixative of the present invention, when the formaldehyde is provided in the form of 10 percent (by volume) formalin, are shown in Table 3, where all concentrations and quantities are understood to be modified by the word “about”:

TABLE 3

| | CONCENTRATION | |
|---|---|---|
| COMPONENT | OR QUANTITY | UNITS |
| Formalin (10 percent, | 2.7 to 16 | Volume percent^{A} |
| by volume)^{C} | | |
| Ketone | 10 to 30 | Volume percent^{A} |
| Alcohol | 25 to 45 | Volume Percent^{A} |
| Water | 10 to 60 | Volume Percent^{A} |
| Sodium Phosphate Monobasic | 0.05 to 0.34 | Grams/1000 ml^{B} |
| Sodium Phosphate Dibasic | 0.2 to 1.2 | Grams/1000 ml^{B} |
| (Anhydrous) | | |
| Sodium Chloride | 1.1 to 6.8 | Grams/1000 ml^{B} |

^{A}Based on the total volume of the formalin, ketone, alcohol and water
^{B}Based on the total volume of the formalin, ketone, alcohol, and water being 1000 milliliters
^{C}Provided as a combination containing (1) 10 volume percent of an aqueous solution containing about 37 to 40 volume percent formaldehyde, about 11 to about 14 volume percent methanol, and the balance water and (2) 90 volume percent water.

Still more exemplary formulation options for the biological fixative of the present invention, when the formaldehyde is provided in the form of 10 percent (by volume) formal in, are shown in Table 4, where all concentrations and quantities are understood to be modified by the word “about”:

TABLE 4

| | CONCENTRATION | |
|---|---|---|
| COMPONENT | OR QUANTITY | UNITS |
| Formalin (10 percent, | 6.6 to 9.3 | Volume percent^{A} |
| by volume)^{C} | | |
| Ketone | 18 to 22 | Volume percent^{A} |
| Alcohol | 34 to 38 | Volume Percent^{A} |
| Water | 31 to 41 | Volume Percent^{A} |
| Sodium Phosphate Monobasic | 0.16 to 0.18 | Grams/1000 ml^{B} |
| Sodium Phosphate Dibasic | 0.58 to 0.6 | Grams/1000 ml^{B} |
| (Anhydrous) | | |
| Sodium Chloride | 3.2 to 3.6 | Grams/1000 ml^{B} |

^{A}Based on the total volume of the formalin, ketone, alcohol and water
^{B}Based on the total volume of the formalin, ketone, alcohol, and water being 1000 milliliters
^{C}Provided as a combination containing (1) 10 volume percent of an aqueous solution containing about 37 to 40 volume percent formaldehyde, about 11 to about 14 volume percent methanol, and the balance water and (2) 90 volume percent water.

A biological fixative with a formulation falling within the parameters provided in Table 4 may be obtained as FROZFIX® biological fixative from Newcomer Supply of Middleton, Wis.

The components of the biological fixative may be further characterized in terms of component ratios. For example, in the biological fixative, the volumetric ratio of the alcohol to the ketone may generally range from as low as about 0.8:1 to as high as about 4.5:1. In some embodiments of the biological fixative, the volumetric ratio of the alcohol to the ketone in the biological fixative may range from as low as about 1.5:1 to as high as about 2.1:1. Still further, in some embodiments, the volumetric ratio of the alcohol to the ketone in the biological fixative may be about 1.8:1. Also, in the biological fixative, the volumetric ratio of the alcohol to the aldehyde may generally range from as low as about 41.5:1 to as high as about 450:1. In some embodiments of the biological fixative, the volumetric ratio of the alcohol to the aldehyde may range from as low as about 97:1 to as high as about 152:1. Still further, in some embodiments, the volumetric ratio of the alcohol to the aldehyde in the biological fixative may be about 120:1

As noted above, the formalin employed in the biological fixative of the present invention may, for example, be neutral buffered ten volume percent formalin solution containing about ten volume percent of a commercially available aqueous formaldehyde solution (containing about 37 to about 40 volume percent formaldehyde, about 11 to about 14 volume percent methanol, and water to make 100 volume percent) and water to make 100 volume percent of the neutral buffered ten volume percent formalin solution. Also, the ethanol employed in the biological fixative of the present invention may, for example, be a commercially-obtained research grade of ethanol containing about 95 volume percent, or more, ethanol, about 2 volume percent, or less, of a denaturant (such as methanol), and the balance water. Next, the acetone employed in the biological fixative of the present invention may, for example, be a reagent grade of acetone containing about 99 volume percent, or more, acetone, about 1.0 volume percent, or less, water, and the balance minor amounts of other polar solvents.

Rinse aids are employed in various protocols described herein. One rinse aid described herein is a modified form of Tris-buffered saline (also referred to herein as “TBS-Modified” and as “Tris-Buffered Saline, Modified”). Tris-Buffered Saline, Modified may be obtained from Newcomer Supply of Middleton, Wis. Alternatively, the Tris-Buffered Saline, Modified, may be prepared by combining and mixing together (1) 250 milliliters of a 0.2 molar solution (in water) of Tris(hydroxymethyl)aminomethane, (2) 385 milliliters of a 0.1 molar solution (in water) of hydrochloric acid, (3) 8.5 grams of ACS grade Sodium Chloride, and (4) a sufficient quantity of water (q.s. to one liter) to bring the total volume of the Tris-Buffered Saline, Modified, to one liter. In general, it is expected, the TBS-Modified will have a pH ranging from as low as about 7.6 to as high as about 7.8 at a temperature of 25° C.

Another rinse aid described herein is a modified form of Tris-buffered saline with TWEEN® 20 surfactant (also referred to herein as “TBS-T-Modified” and as “Tris-Buffered Saline plus Tween, Modified”). Tris-Buffered Saline plus Tween, Modified may be obtained from Newcomer Supply of Middleton, Wis. Alternatively, the Tris-Buffered Saline plus Tween, Modified, may be prepared by combining and mixing together (1) 250 milliliters of a 0.2 molar solution (in water) of Tris(hydroxymethyl)aminomethane, (2) 385 milliliters of a 0.1 molar solution (in water) of hydrochloric acid, (3) 8.5 grams of ACS grade Sodium Chloride, (4) three drops of lab grade TWEEN® 20 surfactant, and (5) and a sufficient quantity of water (q.s. to one liter) to bring the total volume of the Tris-Buffered Saline plus Tween, Modified, to one liter. In general, it is expected, the TBS-T-Modified will have a pH ranging from as low as about 7.6 to as high as about 7.8 at a temperature of 25° C.

Tris(hydroxymethyl)aminomethane crystals that may be used to prepare the 0.2 molar solution (in water) of Tris(hydroxymethyl)aminomethane are available from American International Chemical, Inc. of Framingham, Mass. Hydrochloric acid (0.1 M in water) may be obtained from Carolina Biological Supply Company of Burlington, N.C. ACS grade Sodium Chloride may be obtained from Morton Salt Co. a division of Rohm & Haas Co., Inc. of Philadelphia, Pa. Lab grade TWEEN® 20 surfactant may be obtained from Sigma-Aldrich of St. Louis, Mo.

Various benefits arise from use of the biological fixative of the present invention in accordance with the methods of the present invention. For example, the time required from collection of many biological samples to completion of fixation of the biological samples (so the samples are ready for the desired analysis procedure) using the biological fixative in accordance with the procedures of the present invention may be expected to be as little as about one hour, or even less. Many existing procedures for fixing biological samples, particularly currently popular ones that involve embedding the biological sample with paraffin, commonly require twenty-four hours, or more, from collection of the biological sample through fixation of the biological sample and subsequent preparation until the sample is ready for the desired analysis procedure. The speed with which the sample may proceed from collection through fixation so the sample is ready for the desired analysis procedure renders the biological fixative and methods of the present invention particularly suitable for rapid intraoperative consultations when a patient remains in surgery and immediately available for further procedures, should the results of intraoperative consultations so dictate.

Other benefits from use of the present invention in accordance with the methods of the present invention relate to morphology stabilization. Morphology stabilization means stabilizing the structure of the biological sample, such as mammalian tissue, in as close proximity to the structure the tissue had when part of the living being, as possible. Use of the biological fixative of the present invention in accordance with the methods of the present invention has been found to beneficially attain superior morphology stabilization. This is particularly true with regard to high fat organs and tissues, such as adipose tissue and brain tissue, that are ordinarily considered some of the most difficult biological samples in which to stabilize morphology.

Still other benefits from use of the present invention in accordance with the methods of the present invention relate to antigenicity stabilization. Antigenicity stabilization means stabilizing the antigens of the biological sample, such as mammalian tissue, so as many of the antigens originally present in the biological sample are both present in the sample and remain accessible for antibody binding and engagement of any desired detection system(s) to the bound antibody. Use of the biological fixative of the present invention in accordance with the methods of the present invention has been found to beneficially attain superior antigenicity stabilization, as compared to many existing stabilization approaches.

Ultimately, the benefits from use of biological fixative of the present invention in accordance with the methods of the present invention are many, as explained above. Further benefits stem from the beneficial combination of the time-saving aspects in combination with good or even excellent morphology stabilization and with good or even excellent antigenicity stabilization. Ultimately, due to the enhanced cellular stabilization achieved from use of the present invention in accordance with the methods of the present invention, improved light microscope visualization of both morphology and antigenicity is obtained. This allows scientists to better understand the morphology and antigenicity of biological samples originally present in the living being where the biological samples originally existed.

The biological fixative of the present invention does not require any particular time-consuming or complex preparation steps or procedures. Rather, the components of the biological fixative, particularly when employing a commercially-available aqueous formalin composition, may be easily prepared by merely combining and blending the components together. No particular component addition sequence is believed necessary. For example, the aldehyde (as, for example, neutral buffered formalin (an aqueous solution)), ketone, and alcohol may be combined with water, in no particular addition sequence, in a suitable mixing container and thereafter uniformly blended together. Following preparation, the biological fixative of the present invention may generally be stored up to about twelve months at room temperature prior to use.

The biological fixatives of the present invention and the fixation methods of the present invention employing the inventive biological fixatives may generally be used on a wide variety of biological samples. The fixation methods of the present invention entail placing the biological fixative in intimate contact with the biological sample.

The biological samples may originate from any living or dead member of the mammalian, reptilian, amphibian, marine, avian, protozoan, invertebrate (anthropods and insects), parasitic, and botanical species. Non-exhaustive examples of suitable mammalian sources of the biological samples may include human, equine, bovine, murine, and canine beings. Non-exhaustive examples of suitable reptilian sources include snakes and alligators. Non-exhaustive examples of suitable marine sources include fish, oysters, scallops, rays, and jellyfish. Non-exhaustive examples of suitable parasitic sources include worms and flagellates. Non-exhaustive examples of suitable botanical sources include plants and microbials, such as protozoa, bacteria, and fungi.

The biological samples may be any substance exhibiting cellularity, which means the state of a tissue, mass, fluid, or other substance with regard to the degree, quality, or condition of cells present in the tissue, mass, fluid, or other substance. The term “cell” means the smallest structural unit of an organism that is capable of independent functioning and consists of one or more nuclei, cytoplasm, or various organelles, all surrounded by a semi-permeable cell membrane. In addition to including whole, intact cells, the biological samples may also include cell fragments, even where whole, intact cells are not present. Furthermore, the biological samples may include cell aggregates.

Some non-exhaustive examples of biological samples suitable for receiving biological fixatives of the present invention and for application of the fixation methods of the present invention employing the inventive biological fixatives include fluid and semi-fluid biological samples, both soft and hard tissues (and fragments thereof); viruses, protozoa (amoebas, ciliates, sporozoans, and the like), parasites (flagellates and the like), bacteria, and fungi. Some non-exhaustive examples of suitable fluid and semi-fluid matter include hematology specimens, such as blood and blood components; medical dialysis fluids, such as fluids resulting from kidney dialysis procedures; bronchial lavage (mucous); secretions from body organs and tissue; scrape-collected or swab-collected substances from tissue linings, such as scrapings or swabs from inside the mouth or throat, from the gastrointestinal tract, and from the vagina (pap smears); gastric fluids; peritoneal fluids; pleural fluids; synovial fluids; spinal fluids; fluids surrounding an organ, such as the heart or brain; fluids surrounding a joint, such as the knee; endocrine fluids; fecal matter; urine, and semen, so long as the collected fluid and semi-fluid matter exhibits cellularity, either living or dead. In addition, biological samples suitable for receiving biological fixatives of the present invention and for application of the fixation methods of the present invention employing the inventive biological fixatives include cultures grown from any fluid or semi-fluid biological samples, grown from any soft and hard tissues (or any fragment thereof), grown from any virus, grown from any protozoa, grown from any bacteria, and grown from any fungi.

Some non-exhaustive examples of suitable soft tissues include organs, such as the liver, kidney, brain, heart, bladder, stomach, intestines, eyes, and lungs; muscle tissue; skin; nerves; vessels, such as the urethra, blood vessels, and bile ducts; endocrine tissue; adenoid tissues; lymphoid tissues; tonsils; and adipose tissue, such as breast tissue, so long as so long as the collected soft tissue exhibits cellularity, either living or dead. Some non-exhaustive examples of suitable hard tissues include bones, teeth, cartilage, tendons, ligaments, hair, and fingernails, so long as the collected hard tissue exhibits cellularity, either living or dead.

Some non-exhaustive examples of suitable viruses include Cytomegalovirus, which has been linked to formation of brain tumors; Herpes simplex I and II viruses; Adenovirus; Hepatitis C Virus; Epstein Barr Virus; and Papilloma virus. Some non-exhaustive examples of suitable protozoa include amoeba, such as Entamoeba sp., Giardia sp., and Isopora. Some non-exhaustive examples of suitable parasites include flagellates, such as Trichomonas sp. Some non-exhaustive examples of suitable bacteria include acid fast bacteria, such as Mycobacterium sp and the like; gram positive bacteria, such as Streptococcus sp., Staphylococcus sp., Actinomyces sp, Bacillus sp., and the like; and gram negative bacteria, such as Escherichia sp., Salmonella sp., Klebsiella sp., Pseudomonas sp, Neisseria sp., Proteus sp., Enterbacter sp., and the like. Some examples of suitable fungi include Candida sp., Pneumocystis sp., Histoplasma sp., Coccidiodes sp., Blastomyces sp., and the like.

The biological fixative of the present invention may be employed to fix biological samples in combination with a variety of different pre-fixation procedures. Various protocols relating to freezing, conditioning, and sectioning biological samples are provided below as Protocol One through Protocol Six in the PROCEDURAL PROTOCOLS section of this document. Though Protocols One through Six are drafted in terms of “biological tissue” terminology, biological samples other than biological tissue may be also fixed using the biological fixative of the present invention and are likewise encompassed within the fixation method of the present invention. Thus, biological samples, in addition to biological tissue, may be subjected to any of Protocols One through Protocol Six, unless otherwise indicated herein.

For example, not all biological samples require sectioning in order to be employed in analytical procedures (such as those described in Protocols Seven through Ten below) or in procedures designed to capture or retrieve particular components of the biological samples. These biological samples not requiring sectioning may, along with biological samples amenable to sectioning, be employed in analytical procedures (such as those described in Protocols Seven through Ten below), or procedures designed to capture or retrieve particular components of the biological samples. Protocols Seven through Ten below, while drafted in terms of biological samples amenable to sectioning, may nonetheless be performed on biological samples not requiring sectioning.

Some examples of biological samples, such as biological tissues, are often amenable to sectioning because the biological sample, such as biological tissue, is originally relatively thick dimensionally prior to sectioning. On the other hand, other biological samples, such as fluid biological samples, some semi-fluid biological samples, cultures, viruses, protozoa, bacteria, fungi, and even some biological tissues (some hard biological tissues, such as hair) when placed on a suitable support substrate, such as a slide, may be relatively thin dimensionally, such as on the order of a few microns thick. Such dimensionally thin types of biological samples typically do not require sectioning prior to being employed in analytical procedures (such as those described in Protocols Seven through Ten below) or in procedures designed to capture or retrieve particular components of the biological samples.

Fluid biological samples, semi-fluid biological samples, cultures, viruses, protozoa, bacteria, fungi, and biological tissues (such as hair) that do not require sectioning prior to being employed in analytical procedures (such as those described in Protocols Seven through Ten below), or in procedures designed to capture or retrieve particular components of the biological samples, may still be frozen in accordance with Freezing Protocols One through Three and fixed in accordance with Fixation Protocol Six in accordance with the present invention. However, such biological samples not requiring sectioning need not be subjected to Sectioning Protocol Four. Likewise, biological samples not requiring sectioning need not necessarily receive any frozen processing media and therefore may be excluded from Conditioning Protocol Five where frozen processing media is removed from biological sections following sectioning.

Instead, such biological samples not requiring sectioning may be frozen in accordance with any of Freezing Protocols One through Three after being applied or attached to a suitable support substrate, such as a slide. Therefore, for biological samples not requiring sectioning, any details provided herein relating to sectioning or to application of frozen processing media may be skipped, and the frozen biological sample resulting from any of Freezing Protocols One through Three may, after being equilibrated to a temperature of about −20° C., as described subsequently, optionally proceed directly to Step 1 of Fixation Protocol Six if no frozen processing media is employed. Thereafter, any references to biological section, biological tissue section, and the like in Protocols Six through Ten may instead be considered as references to biological sample, for biological samples not requiring sectioning and not containing any frozen processing media.

As noted above, biological samples not requiring sectioning prior to being employed in analytical procedures (such as those described in Protocols Seven through Ten below), or in procedures designed to capture or retrieve particular components of the biological samples, are samples that, when placed on a suitable support substrate, such as a slide, are relatively thin dimensionally, such as on the order of a few microns thick. Such biological samples not requiring sectioning may, as noted above, include biological samples such as fluid biological samples, semi-fluid biological samples, cultures, viruses, protozoa, bacteria, fungi, and some biological tissues (such as hair). Fluid biological samples such as blood and others listed herein may be applied as smears a few microns thick on examination slides using conventional laboratory techniques, in preparation for freezing in accordance with any of Freezing Protocols One through Three. For example, a smear of the fluid biological sample may be prepared by placing a few drops of the fluid biological sample on the slide (the examination slide) and then running the edge of another slide along the examination slide to distribute the fluid biological sample drops as a thin uniform layer a few microns thick on the examination slide. Also, viruses, protozoa, bacteria, and fungi may be distributed in a neutral solution that is thereafter handled like a fluid biological sample and applied as a smear on an examination slide.

Some semi-fluid biological samples may have a sufficiently thin consistency to form a thin layer of the semi-fluid biological sample on an examination slide, while other semi-fluid biological samples may be too thick in consistency to allow formation of a thin layer of the semi-fluid biological sample. Semi-fluid biological samples that are too thick in consistency to allow formation of a thin layer of the semi-fluid biological sample a few microns thick on the examination slide may be diluted with an appropriate diluent, such as saline solution, in an attempt to allow formation of a sufficiently thin layer of the semi-fluid biological sample on the examination slide. Alternatively, semi-fluid biological samples which are too thick in consistency to allow formation of a thin layer of the semi-fluid biological sample a few microns thick on the examination slide may be subjected to sectioning; either with or without addition of an inert thickening agent, and therefore handled like the biological tissues described in Protocols One through Six.

Cultures are another example of a biological sample that do not require sectioning prior to being employed in analytical procedures (such as those described in Protocols Seven through Ten below) or in procedures designed to capture or retrieve particular components of the biological samples. Cultures may be grown from any fluid or semi-fluid biological samples, grown from any soft and hard tissues (or any fragment thereof), grown from any virus, grown from any protozoa, grown from any bacteria, and grown from any fungi. The culture may be grown in a petri dish that includes a coverslip in conventional fashion so the culture grows on the coverslip. The coverslip which includes the culture distributed as a thin uniform layer a few microns thick thereafter serves as an examination slide. Alternatively, the culture may be grown using a chamber slide that is transformable into an examination slide by removing the chamber. Suitable chamber slides are available as LAB-TEC CHAMBER SLIDES from Nalge Nunc International of Rochester, N.Y. and from various laboratory supply companies, such as Cole-Parmer Instrument Company of Vernon Hills, Ill. Cultures grown using chamber slides typically exist as a monolayer that is only a few microns thick on the examination slide.

Prior to being fixed in accordance with the present invention, biological samples amenable to sectioning may be frozen and then sectioned per any of Protocols One through Three in combination with Protocol Four. In one exemplary approach, the sectionable biological sample is frozen per any of Protocol One, Protocol Two, or Protocol Three; the frozen biological sample is then sectioned per Protocol Four below; the frozen biological section is then conditioned per Protocol Five below in preparation for fixation; and the conditioned biological section is then fixed using the biological fixative of the present invention, per Protocol Six.

It is permissible to freeze the frozen biological samples or the frozen biological sections to lower temperatures than initially attained when the biological sample is first frozen, for short term or long term storage between performance of different protocols, as detailed in the PROCEDURAL PROTOCOLS section of this document. Procedural details about recovering the frozen biological samples or the frozen biological sections from such more highly frozen conditions (colder temperatures) are provided in the various Protocols.

For example, if the frozen biological sample or the frozen biological section contains frozen processing media and is held at a temperature ranging between −20° C. and −140° C., procedural details for conditioning the sample to a warmer condition in preparation for fixation with the inventive biological fixative per Protocol Six are provided in Protocol Five. For those frozen biological samples or frozen biological sections not containing any frozen processing media, but still held at a temperature ranging between −20° C. and −70° C., the frozen biological sample or frozen biological section may be placed in a freezer maintained at a temperature of about −20° C. for a minimum of about two hours, prior to proceeding with Step 1 of Fixation Protocol Six. Likewise, for those frozen biological samples or frozen biological sections not containing any frozen processing media, but still held at a temperature ranging between −70° C. and −140° C., the frozen biological sample or frozen biological section may be placed in a freezer maintained at a temperature of about −20° C. for a minimum of about 24 hours, prior to proceeding with Step 1 of Fixation Protocol Six.

Also, in preparation for fixation of biological sections in accordance with the present invention, a procedure for removing any frozen processing media used prior to initial freezing is provided in Protocol Five. Prior to Protocol Five, a fresh biological sample, such as a fresh biological tissue section, is wetted with frozen processing media so the frozen processing media infiltrates the fresh biological sample. The frozen processing media helps stabilize the morphology of the biological sample as the biological sample is being frozen. If the frozen biological sample is later sectioned, the frozen processing media also facilitates cutting of the biological sample while the biological sample is in the frozen state. Though not bound by theory, the frozen processing media is thought to stabilize the morphology of the biological tissue sample by increasing the viscosity within the sample at temperatures below 0° C. and thereby decreasing the mobility of water molecules in the sample at temperatures below 0° C. Limiting the mobility of water molecules is thought to inhibit, or even prevent, the water molecules present in the sample from forming ice crystal nuclei, and ice crystal formation is thereby believed inhibited. Due to its protective effect on morphology while the biological sample is being frozen, the frozen processing media may also be characterized as a cryoprotectant.

One suitable example of the frozen processing media is Tissue-Tek® OCT solution that is available from Sakura Finetek of Torrance, Calif. Another suitable example of the frozen processing media is the CRYO-GEL® product available from Instrumedics, Inc. of St. Louis, Mo. Other substances that may serve as the frozen processing media are believed to include aqueous solutions of dimethyl sulphoxide (DMSO), glycerol, ethylene glycol, dimethyl formamide (DMF), and aqueous solutions of any of these in any combination. Other substances that are believed suitable for use as the frozen processing media include polyvinyl pyrrolidone (PVP), dextran, hydroxyethyl starch, sucrose, and aqueous solutions of any of these in any combination, though these substances are thought to have less infiltration ability than aqueous solutions of DMSO, glycerol, ethylene glycol, and DMF. Therefore, it is thought useful to include one or more of DMSO, glycerol, ethylene glycol, and DMF in combination with one or more of PVP, dextran, hydroxyethyl starch, or sucrose in aqueous solution to support adequate infiltration into the biological tissue sample. For example, a solution of 0.5 mol sucrose and 3.5 mol DMSO in one liter of water is known to adequately infiltrate a biological tissue sample at room temperature prior to freezing.

After application of the frozen processing media to the biological sample, such as the biological tissue sample, the treated biological sample is rapidly frozen. For example, the treated biological sample may be immersed in liquid nitrogen for about fifteen (15) to about thirty (30) seconds to rapidly freeze the treated biological sample to a temperature of about −20° C. Other approaches to rapidly freezing the treated biological sample to a temperature of about −20° C. may be substituted in place of the liquid nitrogen immersion approach. The rapid freezing in liquid nitrogen helps prevent ice crystal formation in the treated biological sample and consequent damage to the treated biological sample. The frozen treated biological sample, while still frozen, is sectioned using a suitable apparatus, such as a microtome located in a cryostat that maintains the frozen state of the biological sample during the sectioning process. Again, the previously applied frozen processing media facilitates cutting, such as sectioning, of the biological sample while the biological sample is in the frozen state.

Protocol Five entails conditioning to remove the frozen processing media from frozen biological sections, such as frozen biological tissue sections, following cutting of the frozen treated biological sample to form frozen biological sections. The frozen processing media is preferably removed from the biological section prior to fixation of the biological section because fixation of the biological section using the biological fixative of the present invention in accordance with the fixation method of the present invention has surprisingly been discovered to result in enhanced antigenicity stability and enhanced morphologic stability following fixation, particularly if the frozen processing media is removed from the biological section prior to fixation of the biological section. According to Protocol Five, the frozen treated biological section, such as the frozen treated biological tissue section, is stabilized at a temperature of about −20° C. for a period ranging from about two hours (if previously stored at −20° C. and −70° C.) to at least about 24 hours (if previously stored at −70° C. and −140° C.). The frozen treated biological section is then warmed to room temperature for a short period of time and is then sequentially immersed in an appropriate solvent of the frozen processing media to facilitate removal of the frozen processing media from the biological section.

Ethanol and a ketone, such as acetone, are two exemplary solvents of the Tissue-Tek® OCT solution. Thus, for example, the treated biological sections may be sequentially immersed in an aqueous ethanol solution (about 95 volume percent ethanol and about 5 volume percent water) with continuous dips for twenty, or more, consecutive dips to initiate removal of the Tissue-Tek® OCT solution. Thereafter, the treated biological sections may be sequentially immersed in a solution containing one part by weight aqueous ethanol solution (about 95 volume percent ethanol and about 5 volume percent water) and one part by weight acetone with continuous dips for twenty, or more, consecutive dips to further facilitate removal of the equivalent frozen processing media, such as the Tissue-Tek® OCT solution. Thereafter, the resulting conditioned biological sections may be fixed (stabilized) using the biological fixative of the present invention in accordance with the fixation method of the present invention, such as that detailed in Fixation Protocol Six below.

Fixation of biological samples using the biological fixative of the present invention is straightforward. The fixative is placed in intimate contact with the fixative of the present invention, such as FROZFIX® biological fixative available from Newcomer Supply of Middleton, Wis. The biological fixative may be applied to the biological sample in any conventional fashion, such as by dipping or otherwise immersing the biological sample in the biological fixative. Also, the biological fixative may be sprayed or poured onto the biological sample.

For example, the biological sample, such as a frozen, non-sectioned, biological sample or a biological section, may be dipped into the room temperature biological fixative of the present invention, such as the FROZFIX® biological fixative. If a routine dye chemistry procedure will be performed on the fixed biological sample (or fixed biological section), it is thought the biological sample (or conditioned biological section) may need to remain immersed in the fixative of the present invention for only about two to about five minutes. If a molecular assay, immunohistochemistry procedure, or immunofluorescence procedure will be performed on the fixed biological sample (or conditioned and fixed biological section), it is thought the biological sample (or conditioned biological section) should generally remain immersed in the fixative of the present invention for about twenty-five to about forty-five minutes.

After the fixed biological sample (or fixed biological section) is removed from the fixative of the present invention, the fixed biological sample (or fixed biological section) may be rinsed in a suitable rinse aid, such as room temperature Tris-Buffered Saline plus Tween, Modified (TBS-T-Modified) (See Fixation Protocol Six below, for example), prior to instituting the desired analysis procedure. Alternatively, after the fixed biological sample (or fixed biological section) is removed from the fixative of the present invention, the fixed biological sample (or fixed biological section) may be rinsed in a suitable rinse aid, such as room temperature Tris-Buffered Saline plus Tween, Modified (TBS-T-Modified) and then held in preparation for instituting the desired analysis procedure (See Fixation Protocol Six below, for example).

Biological samples that have been fixed using the biological fixatives of the present invention via the fixation methods of the present invention employing the inventive biological fixatives may generally undergo a wide variety of analytical procedures. These analytical procedures may be designed to identify normal cells, as well as, abnormal cells. Identification of normal cells in a fixed biological sample can help delineate or distinguish the extent of abnormal cells and reduce the portion of a living being subjected to surgical removal or treatment of abnormal cells. These analytical procedures may also be designed to capture or retrieve particular components of the fixed biological sample.

For example, fixed biological samples produced in accordance with the present invention may undergo visual or aided (microscope) observations and analytical procedures (such as H&E staining, as in Protocol Ten) for rapid intraoperative consultations where a patient remains in surgery and is immediately available for further procedures, should the results of intraoperative consultations so dictate. Also, fixed biological samples in accordance with the present invention may undergo molecular biology analysis, such as immunohistochemistry procedures; immunocytochemical procedures; immunofluorescence procedures, such as various procedures incorporating fluorochromes; confocal microscopy procedures; laser capture microdissection procedures; DNA/RNA in situ hybridization procedures; electronmicroscopy procedures (scanning electron microscopy); other DNA or RNA assessment procedures, such as gel electrophoresis; polymerized chain reaction (PCR) amplification; and microarray procedures, such as gene microarray procedures.

Exemplary immunohistochemistry and immunofluorescence procedures may employ one marker (only one primary antibody), two markers (two different primary antibodies), or even three or more markers (three different primary antibodies). Exemplary confocal microscopy procedures include single and multiple marker immunofluorescence procedures. Exemplary laser capture dissection procedures include isolation and/or capture of nucleic acid molecules, such as DNA and/or RNA molecules. Exemplary DNA/RNA in situ hybridization procedures include fluorescent and chromogenic or non-radioactive procedures. Detailed exemplary protocols for immunohistochemistry and immunofluorescence procedures that may be performed on biological samples fixed using fixatives of the present invention in accordance with fixation procedures of the present invention are provided below under the PROCEDURAL PROTOCOLS section of this document. These protocols generally entail engagement of a primary antibody with a target antigen of the fixed biological sample, followed by engagement of a secondary antibody to the engaged primary antibody, followed by binding of an enzymatic label to the engaged secondary antibody to complete creation of a signal composite; chromogen or fluorochrome is then applied to the signal composite and a color reaction occurs that yields a color indicator for positive signals where the target antigen is present in the fixed biological sample.

Though the exemplary protocols for immunohistochemistry, (Protocol Seven), immunofluorescence (Protocols Eight and Nine) and H&E dye chemistry (Protocol Ten) procedures provided below under the PROCEDURAL PROTOCOLS section of this document are described in terms of acting on a biological section, such as a biological tissue section, these exemplary protocols are not limited to action on biological sections or biological tissue sections. Rather, any references to biological section, biological tissue section, and the like in Protocols Six through Ten may instead be considered as references to any biological sample fixed using the biological fixative of the present invention in accordance with the fixation method of the present invention. Thus, Protocols Six through Ten apply equally to biological samples that have been sectioned and to biological samples that have not been sectioned. Also, Protocols Six through Ten, besides being applicable to biological tissue (both soft and hard) samples, are also applicable to other biological samples encompassed by the fixation method of the present invention, such as fluid biological samples, semi-fluid biological samples, cultures, viruses, protozoa, bacteria, fungi, and the like.

In addition, beyond the exemplary protocols for immunohistochemistry and immunofluorescence procedures provided below under the PROCEDURAL PROTOCOLS, other analytical procedures, visualization procedures, intraoperative procedures, and the like, such as laser capture dissection procedures, polymerized chain reaction (PCR) procedures, proteometric procedures, gene microarray procedures, DNA/RNA in situ hybridization procedures, and others not specifically named herein are applicable to both biological tissue (both soft and hard) samples and other biological samples encompassed by the fixation method of the present invention, such as fluid biological samples, semi-fluid biological samples, cultures, viruses, protozoa, bacteria, fungi, and the like. Furthermore, beyond the exemplary protocols for immunohistochemistry and immunofluorescence procedures provided below under the PROCEDURAL PROTOCOLS, other analytical procedures, visualization procedures, intraoperative procedures, and the like, such as laser capture dissection procedures, polymerized chain reaction (PCR) procedures, proteometric procedures, gene microarray procedures, DNA/RNA in situ hybridization procedures, and others not specifically named herein are applicable to both sectioned biological samples and biological samples that need not be sectioned to attain a layer sufficiently thin dimensionally for the particular procedure.

As noted above, multiple primary antibodies may be employed to mark different target antigens on a single biological sample. The general technique entails first (1) assembling a first signal composite of a first target antigen, a first primary antibody, a first secondary antibody, and a first enzymatic label; (2) applying a first signal (color indication) system to the first signal composite; (3) assembling a second signal composite of a second target antigen, a second primary antibody, a second secondary antibody, and a second enzymatic label; and (4) applying the second signal (color indication) system to the second signal composite. Where triple marking is desired, a third signal composite is prepared and a third signal (color indication) system is applied to the third signal composite. An exemplary dual marker analysis is described in detail in Immunofluorescence Protocol Nine below.

In a multiple marker system, the first primary antibody and the second primary antibody will ordinarily be different from each other to allow different antigens to be targeted by the different primary antibodies. Also, the first primary antibody and the second primary antibody will typically be from different animal sources. Furthermore, the first signal (color indication) system and the second signal (color indication) system will ordinarily be different from each other so different targeted antigens yield signals of different colors. For immunofluorescence procedures, two examples of signal systems (fluorochromes) exhibiting different colors are Tyramide Rhodamine (Red) fluorochrome and Tyramide Fluorescein (green) fluorochrome, which may be obtained using Catalog No. NEL702 and Catalog No. NEL701, respectively, from Life Science Products, Inc. of Boston, Mass.

Various primary antibodies may be employed in single or multiple marker versions of immunohistochemistry procedures and immunofluorescence procedures (such as confocal microscopy procedures) performed on fixed biological samples produced in accordance with the present invention, so long as the chosen primary antibody is compatible with the target antigen of the biological sample. For example, neuroendocrine markers, endothelial markers, hematopoietic markers, infectious agents, intermediate filaments, myogenic markers, oncoproteins, prognostic markers, tumor-associated antigens, and other miscellaneous substances may serve as the primary antibody. Some examples of suitable hematopoietic markers that may serve as the primary antibody include cluster defined (CD) antigens, lymphoid/myeloid markers, and immunoglobulins. The primary antibody may come from any suitable animal source. Some common suitable sources of the primary antibody include mice and rabbits. Primary antibodies of these types and sources, such as the various examples of particular primary antibodies, are typically available from most major laboratory supply companies that carry antibodies. As some examples, suitable primary antibodies of these types and sources may generally be obtained from Biogenex Laboratories of San Ramon, Calif., from Innovex BioSciences of Richmond, Calif., and/or from Biocare Medical of Walnut, Calif.

Some suitable examples of neuroendocrine markers that may serve as the primary antibody include Chromogranin A, Neuron specific enolase, Synaptophysin, and Vasoactive intest. polypeptide. Some suitable examples of endothelial markers that may serve as the primary antibody include CD31 (JC/70A), CD34 (QBEnd), and Factor VIII related Ag. Some suitable examples of Cluster defined (CD) antigens of the hematopoietic marker group that may serve as the primary antibody include CD1a (010), CD3 (T-cell) (CP), CD4 (1F6), CD5 (4C7), CD8 (1A5), CD10 (56C6), CD15 (LeuM1), CD20 (L26) pan B-cell, CD21 (1F8), CD23 (1B12), CD30 (Ber-H2) (Ki-1), CD31 (Pecam), D45 (LCA), CD45R0 (UCHL-1), CD45RA (B-cell), CD68 (macrophage) (KP1), and CD79a (B-cell). Some suitable examples of lymphoid/myeloid markers of the hematopoietic marker group that may serve as the primary antibody include bcl-1 (Cyclin D1), bcl-2 oncoprotein, bcl-6 (PG-B6P), Myeloperoxidase, T-cell (OPD4), and T-cell, pan (UCHL-1). Some suitable examples of immunoglobulins of the hematopoietic marker group that may serve as the primary antibody include IgG, IgM, IgA, IgD, κ light chains, and λ light chains. Some suitable examples of infectious agents that may serve as the primary antibody include Adenovirus (20/11, 2/6), Cytomegalovirus (IE, IE₂, LMP), Epstein Barr virus (LMP), Epstein Barr virus (EBNA), Helicobacter pylori, Herpes simplex I & II, Human papilloma virus, and Hepatitis C Virus. Some suitable examples of intermediate filaments that may serve as the primary antibody include GFAP (Glial), Keratin 5/6, Keratin 7, Keratin 20, Keratin (Broad spectrum) (AG1/AE3/PCK26), Keratin 10, Keratin, HMW (34BE12) (Ker903), Keratin, HMW (AE3), Keratin, LMW (AE1), Keratin LMW (MAK-6), Keratin, LMW (CAM 5.2), Neurofilaments (neural) 2F11), Vimentin (3B4). Some suitable examples of myogenic markers that may serve as the primary antibody include Actin, muscle specific (HUCI-1), x Actin, α-smooth muscle (1A4), and Desmin.

Some suitable examples of oncoproteins that may serve as the primary antibody include C-erbB₂ (HER-2/neu) and p53 (Bp53-11). Some suitable examples of PROGNOSTIC MARKERS that may serve as the primary antibody include K1-67 (MIB-1) and PCNA (PC10). Some suitable examples of tumor-associated antigens that may serve as the primary antibody include CA19.9, CA 125 (OC125), Carcinoembryonic antigen (CEA), Carcinoembryonic antigen (CEA), Epithelial membrane ag. (EMA), Factor XIIIa, HMB45 (melanoma), Prostate specific ag (PSA), Prostate specific acid phos., S-100 (4C4.9), and TTF-1. Some other examples of suitable primary antibodies include miscellaneous markers, such as DNA/mRNA insitu hybrid, DNA ISH-HPB 6/11/18, DNA ISH-HPV 16/18, DNA ISH-CMV, DNA ISH-EBV (EBER), and other markers, such as FCR 4, FCR 5, MHC Class II (mouse tissues), B1B (rat tissues), and K1-A (rat tissues).

Various secondary antibodies may employed in single or multiple marker versions of immunohistochemistry procedures and immunofluorescence procedures (such as confocal microscopy procedures) performed on fixed biological samples produced in accordance with the present invention, so long as the chosen secondary antibody is compatible with the primary antibody to be engaged by the secondary antibody. The choice of secondary antibody will depend on the animal source of the primary antibody, since the animal source of the primary antibody will need to differ from the animal source of the secondary antibody. For example, if the source of the primary antibody is mouse, the source of the secondary antibody should be something other than mouse, such as goat anti-mouse. Some examples of suitable secondary antibodies available from Biocare Medical of Walnut, Calif. (along with the Biocare Medical catalog number) are provided in Table 5 below:

TABLE 5

| | Biocare Medical |
|---|---|
| Name of Biotinylated Secondary Antibody | Catalog No. |
| Universal Goat Link | GU600G |
| Universal Goat Link | GU600H |
| Goat Anti-Mouse IgG (Hrs, bov, hum abs) | GM601G |
| Goat Anti-Mouse IgG (Hrs, bov, hum abs) | GM601H |
| Goat Anti-Rabbit IgG (Human Absorbed) | GR602G |
| Goat Anti-Rabbit IgG (Human Absorbed) | GR602H |
| Goat Anti-Mouse IgM | GM603G |
| Goat Anti-Mouse IgG (Rat Absorbed) | GM606G |
| Goat Anti-Mouse IgG (Rat Absorbed) | GM606H |
| Goat Anti-Rat IgG (Mouse Absorbed) | GR607G |
| Goat Anti-Rat IgG (Mouse Absorbed) | GR607H |
| Goat Anti-Rabbit IgG (Mouse & Rat Adsorbed) | GR608G |
| Goat Anti-Rabbit IgG (Mouse & Rat Absorbed) | GR608H |
| Mouse Anti-Goat IgG | MG610G |
| Mouse Anti-Goat IgG | MG610H |
| Rabbit Anti-Sheep | Not Available |

The list of some suitable secondary antibodies provided in Table 5 above is not exhaustive and other suitable secondary antibodies are available. These and other suitable secondary antibodies may generally be obtained from Biocare Medical of Walnut Creek, Calif., from Biogenex Laboratories of San Ramon, Calif., and from Innovex BioSciences of Richmond, Calif.

Protocols for immunohistochemistry procedures, for both immunofluorescence procedures and confocal microscopy procedures, and for H & E dye staining using biological samples that have been fixed using the biological fixative of the present invention via the fixation methods of the present invention are described herein (See Protocols Seven through Ten below, for example). Those skilled in the art of intrasurgical consultations and molecular biology techniques would be able to adapt molecular biology techniques to incorporate biological samples that have been fixed using the biological fixative of the present invention via the fixation methods of the present invention. Therefore, the present invention is believed to extend to any intrasurgical consultation procedure and any molecular biology technique incorporating biological samples that have been fixed using the biological fixative of the present invention via the fixation methods of the present invention, even though specific protocols for each and every available intrasurgical consultation procedure and any molecular biology technique incorporating biological samples are not necessarily provided herein.

### Procedural Protocols

Various procedural protocols that may be used on biological samples in accordance with the present invention are described below. The procedural protocols described herein are exemplary only and are not an exhaustive list of procedural protocols that may be used on biological samples fixed in accordance with the present invention, as those of ordinary skill in the art will recognize.

Two different buffered rinse solutions referred to repeatedly in many of these protocols are noted below along with preparation and source information:

Tris-Buffered Saline, Modified (TBS-Modified)
Tris-Buffered Saline, Modified may be obtained from Newcomer Supply of Middleton, Wis. Alternatively, the Tris-Buffered Saline, Modified, may be prepared by combining and mixing together (1) 250 milliliters of a 0.2 molar solution (in water) of Tris(hydroxymethyl)aminomethane, (2) 385 milliliters of a 0.1 molar solution (in water) of hydrochloric acid, (3) 8.5 grams of ACS grade Sodium Chloride, and (4) a sufficient quantity of water (q.s. to one liter) to bring the total volume of the Tris-Buffered Saline, Modified, to one liter. In general, it is expected, the TBS-Modified will have a pH ranging from as low as about 7.6 to as high as about 7.8 at a temperature of 25° C. Tris(hydroxymethyl)aminomethane crystals that may be used to prepare the 0.2 molar solution (in water) of Tris(hydroxymethyl)aminomethane are available from American International Chemical, Inc. of Framingham, Mass. Hydrochloric acid (0.1 M in water) may be obtained from Carolina Biological Supply Company of Burlington, N.C. ACS grade Sodium Chloride may be obtained from Morton Salt Co. a division of Rohm & Haas Co., Inc. of Philadelphia, Pa.

Tris-Buffered Saline-Tween-Modified (TBS-T-Modified)
Tris-Buffered Saline plus Tween, Modified may be obtained from Newcomer Supply of Middleton, Wis. Alternatively, the Tris-Buffered Saline plus Tween, Modified, may be prepared by combining and mixing together (1) 250 milliliters of a 0.2 molar solution (in water) of Tris(hydroxymethyl)aminomethane, (2) 385 milliliters of a 0.1 molar solution (in water) of hydrochloric acid, (3) 8.5 grams of ACS grade Sodium Chloride, (4) three drops of lab grade TWEEN® 20 surfactant, and (5) and a sufficient quantity of water (q.s. to one liter) to bring the total volume of the Tris-Buffered Saline plus Tween, Modified, to one liter. In general, it is expected, the TBS-T-Modified will have a pH ranging from as low as about 7.6 to as high as about 7.8 at a temperature of 25° C.
Tris(hydroxymethyl)aminomethane crystals that may be used to prepare the 0.2 molar solution (in water) of Tris(hydroxymethyl)aminomethane are available from American International Chemical, Inc. of Framingham, Mass. Hydrochloric acid (0.1 M in water) may be obtained from Carolina Biological Supply Company of Burlington, N.C. ACS grade Sodium Chloride may be obtained from Morton Salt Co. a division of Rohm & Haas Co., Inc. of Philadelphia, Pa. Lab grade TWEEN® 20 surfactant may be obtained from Sigma-Aldrich of St. Louis, Mo.

### Freezing Tissue Samples

Freezing Protocols One, Two, and Three below employ liquid nitrogen or dry ice to freeze biological tissue samples. Biological tissue samples are preferably not frozen by being placed directly in a freezer maintained at a temperature ranging between approximately −20° C. and −70° C., since this will typically result in formation of an abundance of ice crystals in the tissue sample and consequently an abundance of tissue sample damage due to the ice crystals. Such abundant ice crystal damage will typically be expected to make sections of the frozen tissue sample unsuitable for procedures such as DNA and RNA In Situ, Confocal Microscopy, and Laser Capture Microdissection procedures. For example brain tissue frozen in this manner (by being placed directly in a freezer maintained at a temperature ranging between approximately −20° C. and −70° C.) will typically exhibit numerous gaps within the tissue sections which makes analysis difficult, if not practically impossible.

Protocol One:

1. Provide a one (1) centimeter by one (1) centimeter square of a three (3) millimeter thick polystyrene (or equivalent) film as a sample support. The polystyrene film helps prevent the biological tissue sample from bending during freezer storage.
2. Cut a sample of the biological tissue to have approximate dimensions of one (1) centimeter by one (1) centimeter by two (2) centimeters.
3. Pour approximately two (2) milliliters of Tissue-Tek® OCT solution (available from Sakura Finetek of Torrance, Calif.), or an equivalent frozen processing media for frozen tissue specimens, onto the polystyrene sample support. The OCT solution facilitates cutting of the biological tissue sample and helps stabilize the morphology of the sample as the sample is being frozen.
4. Lay the biological tissue sample down on the polystyrene sample support in the desired orientation, and pour two (2) more milliliters of the Tissue-Tek® OCT, or equivalent frozen processing media, on top of the tissue so all exposed surfaces of the biological tissue sample are wetted with the frozen processing media.
5. Fashion an immersion tool from metal wire or equivalent material with a loop and a handle section, so the sample support bearing the biological tissue sample can rest on the loop.
6. While holding the handle of the immersion tool, immerse the biological tissue sample and the polystyrene sample support into liquid nitrogen for about fifteen (15) to about thirty (30) seconds to rapidly freeze the biological tissue sample to a temperature of about −20° C. The rapid freezing in liquid nitrogen helps prevent ice crystal formation in the tissue sample and consequent damage to the tissue sample.
7. If the frozen biological sample is to be stored prior to subsequent processing, immediately place the sample support bearing the frozen biological tissue sample in a cassette that is then placed in a freezer maintained at a temperature ranging between −70° C. and −140° C. For long term storage greater than about two months, tightly wrap the cassette in aluminum foil before the cassette is placed in the freezer.
8. Rather than storing the frozen biological tissue sample in the freezer, the sample support bearing the frozen biological tissue sample may be placed in a cassette and sectioned immediately per an appropriate sectioning procedure, such as the sectioning procedure of Protocol Four detailed below.
   Protocol Two:

If the frozen biological tissue sample is to be stored in the frozen state for more than about two months, this Freezing Protocol Two is preferred by some scientists over Freezing Protocol One, since this Freezing Protocol Two avoids use of any frozen processing media, such as the OCT solution. Some scientists think frozen processing media, such as the OCT solution, may cause chemical degradation of the biological tissue sample during longer term freezer storage. Therefore, when conducting procedures on biological samples that do not require sectioning, such as certain RNA detection procedures, and thereafter saving the samples for further study where further staining procedures are not anticipated to occur, scientists will typically employ a procedure, such as this Freezing Protocol Two, that avoids use of frozen processing media, such as the OCT solution. Beneficially, the rapid freezing of this Freezing Protocol Two, like the rapid freezing employed in Freezing Protocol One, helps prevent ice crystal formation in the tissue sample and consequent damage to the tissue sample.
1. Cut a sample of the biological tissue to have approximate dimensions of one and a half (1.5) centimeters by one and a half (1.5) centimeters by three tenths (0.3) centimeters. Larger tissue samples can warp during freezing; cutting to these approximate dimensions can help minimize or avoid this warping issue.
2. Provide a small zipper-locking plastic bag designed for immersion into liquid nitrogen. Bags of this type are common items in most pathology laboratories.
3. Place the cut sample of the biological tissue in the small zipper-locking plastic bag and close the zipper, while being careful to minimize excess air present in the bag upon closure.
4. Immerse the plastic bag holding the cut biological tissue sample into liquid nitrogen for about fifteen (15) to about thirty (30) seconds to rapidly freeze the tissue sample to a temperature of about −20° C. The rapid freezing in liquid nitrogen helps prevent ice crystal formation in the tissue sample and consequent damage to the tissue sample.
5. If the frozen biological tissue sample is to be stored prior to subsequent processing, remove the frozen biological tissue sample from the plastic bag, and place the frozen biological tissue sample in a cassette that is placed in a freezer maintained at a temperature ranging between −70° C. and −140° C. For long term storage greater than about two months, tightly wrap the cassette in aluminum foil before the cassette is placed in the freezer.
6. Rather than storing the frozen biological tissue sample in the freezer, the frozen biological tissue sample may instead be immediately subjected to the sectioned tissue fixation procedure of Protocol Four below. Prior to proceeding to Sectioning Protocol Four, however, frozen processing media should be applied to the frozen biological tissue section, as detailed below in Steps 8 and 9 of this Protocol Two.
7. If the frozen biological tissue sample has been stored in a freezer maintained at a temperature ranging between −70° C. and −140° C., place the frozen biological tissue sample in a freezer maintained at a temperature of about −20° C. for a minimum of about twenty-four (24) hours, prior to applying frozen processing media to the frozen biological tissue section per Steps 8 and 9 of this Protocol Two.
8. Pour approximately two (2) milliliters of Tissue-Tek® OCT solution (available from Sakura Finetek of Torrance, Calif.), or an equivalent frozen processing media for frozen tissue specimens, onto a suitable sample support, such as a polystyrene sample support. The OCT solution facilitates cutting of the biological tissue sample.
9. Lay the frozen biological tissue sample down on the sample support in the desired orientation, and pour two (2) more milliliters of the Tissue-Tek® OCT, or equivalent frozen processing media, on top of the tissue so all exposed surfaces of the frozen biological tissue sample are wetted with the frozen processing media.
10. After application of the Tissue-Tek® OCT, or equivalent frozen processing media, per Steps 8 and 9 of this Protocol Two is complete, proceed to Step 2 of Sectioning Protocol Four.
   Protocol Three:

This Freezing Protocol Three may be employed in place of Freezing Protocol One if liquid nitrogen for rapid freezing is unavailable. However, since the freezing rate obtained using this Freezing Protocol Three is significantly slower than the freezing rate obtained using liquid nitrogen in accordance with Freezing Protocol One, some ice crystal formation is likely to occur in the sample accompanied by consequent damage to the tissue sample due to the ice crystal development.
1. Provide a one (1) centimeter by one (1) centimeter square of a three (3) millimeter thick polystyrene (or equivalent) film as a sample support.
2. Cut a sample of the biological tissue to have approximate dimensions of one (1) centimeter by one (1) centimeter by two (2) centimeters.
3. Pour approximately two (2) milliliters of Tissue-Tek® OCT solution (available from Sakura Finetek of Torrance, Calif.), or an equivalent frozen processing media for frozen tissue specimens, onto the polystyrene sample support.
4. Lay the biological tissue sample down on the polystyrene sample support in the desired orientation, and pour two (2) more milliliters of the Tissue-Tek® OCT, or equivalent frozen processing media, on top of the tissue so all exposed surfaces of the biological tissue sample are wetted with the frozen processing media.
5. Place the polystyrene sample support bearing the biological tissue sample in a cassette, and tightly wrap the cassette in aluminum foil.
6. Place the aluminum foil wrapped cassette bearing the biological tissue sample on a block of dry ice (solid carbon dioxide (CO₂)) for about ten to about fifteen minutes to freeze the biological tissue sample to a temperature of about −20° C.
7. If the frozen biological sample is to be stored prior to subsequent processing, immediately place the aluminum foil wrapped cassette bearing the frozen biological tissue sample in a freezer maintained at a temperature ranging between −70° C. and −140° C.
8. Rather than storing the frozen biological tissue sample in the freezer, the frozen biological tissue sample may be sectioned immediately per an appropriate sectioning procedure, such as the sectioning procedure of Protocol Four detailed below.

### Sectioning Frozen Tissue Samples

Protocol Four:

1. If the frozen biological tissue sample has been stored in a freezer maintained at a temperature ranging between −70° C. and −140° C., place the frozen biological tissue sample in a freezer maintained at a temperature of about −20° C. for a minimum of about twenty-four (24) hours, prior to sectioning.
2. Transfer the frozen biological tissue sample from step 1 of this protocol (or from step 8 of Freezing Protocol One, from Step 10 of Freezing Protocol Two, or from step 8 of Freezing Protocol Three) immediately into a cryostat for sectioning. One example of a suitable cryostat is the Tissue-Tek® Cryo3® microtome/cryostat that may be obtained from Sakura Finetek of Torrance, Calif. The frozen biological sample is held at a temperature of about −20° C. in the cryostat prior to and during sectioning.
3. Section the frozen biological tissue sample in the cryostat according to the procedure accompanying the cryostat. The thickness of each section may generally be about four to about six microns, though the section thickness may be varied up or down, as desired and appropriate for different tissues and particular requirements of different analysis procedures.
4. Place the section of the frozen biological tissue sample onto a positively charged glass slide, onto a silane-coated slide, or an equivalent slide.
5. If the section of the frozen biological sample is to be stored for less than about two months, place the section in a properly sealed slide box and then place the slide box in a freezer maintained at a temperature ranging between −20° C. and −70° C.
6. If the section of the frozen biological sample is to be stored for about two months or more, place the section in a properly sealed slide box and then place the slide box in a freezer maintained at a temperature ranging between −70° C. and −140° C.
7. If the section of the frozen biological sample is to be fixed immediately, continue to Conditioning Protocol Five below.

### Conditioning Frozen Tissue Sections in Preparation for Fixation

Protocol Five:

NOTE: This protocol is applicable only to frozen biological samples that were sectioned, such as via Sectioning Protocol Four above, and is not needed or appropriate for biological samples that have not been sectioned.
   1. If the frozen biological tissue section has been stored in a freezer maintained at a temperature ranging between −70° C. and −140° C., place the frozen biological tissue section in a freezer maintained at a temperature of about −20° C. for a minimum of about 24 hours, prior to proceeding with conditioning in accordance with this protocol.
   2. If the frozen biological tissue section has been stored in a freezer maintained at a temperature ranging between −20° C. and −70° C., place the frozen biological tissue section in a freezer maintained at a temperature of about −20° C. for a minimum of about two hours, prior to proceeding with conditioning in accordance with this protocol.
   3. Acclimate the frozen biological tissue sample from step 1 or step 2 of this protocol (or from step 7 of Sectioning Protocol Four) by laying the slide (tissue side up) containing the frozen section onto a support surface for thirty seconds at room temperature or by warming the bottom of the slide (tissue side up) with natural heat from the index finger for fifteen seconds at room temperature.
   4. Following step 3, sequentially immerse the slide containing the biological tissue section in an aqueous ethanol solution (about 95 volume percent ethanol and about 5 volume percent water) with continuous dips for twenty consecutive dips to facilitate removal of the OCT solution or equivalent frozen processing media from the biological tissue section.
   5. Following step 4, sequentially immerse the slide containing the biological tissue section in a solution containing one part by weight aqueous ethanol solution (about 95 volume percent ethanol and about 5 volume percent water) and one part by weight acetone with continuous dips for twenty consecutive dips to further facilitate removal of the OCT solution or equivalent frozen processing media.

### Fixation of Tissue Sections

Protocol Six:

1. Immerse the slide containing the conditioned biological tissue section obtained in Step 5 of Conditioning Protocol Five (or the slide containing a frozen biological sample free of conditioning media) into the room temperature fixative of the present invention, such as FROZFIX® biological fixative available from Newcomer Supply of Middleton, Wis. If a routine dye chemistry procedure will be performed on the fixed tissue section (or the fixed biological sample), it is thought the conditioned biological tissue section (or the frozen biological sample free of conditioning media) should remain immersed in the fixative of the present invention for only about two to about five minutes. If a molecular assay, immunohistochemistry procedure, or immunofluorescence procedure will be performed on the fixed tissue section (or the fixed biological sample), the conditioned biological tissue section (or the frozen biological sample free of conditioning media) should remain immersed in the fixative of the present invention for about twenty-five to about forty-five minutes.
2. Following Step 1, sequentially immerse the slide containing the fixed biological tissue section (or the fixed biological sample) in room temperature Tris-buffered saline-Tween, Modified (TBS-T-Modified) with continuous dips for ten consecutive dips.
3. Starting with new (fresh) TBS-T-Modified, repeat the immersion procedure of Step 2 (continuous dips for ten consecutive dips) three times; change to new (fresh) TBS-T-Modified after each set of ten dips.
4. After step 3, immerse the slide containing the fixed biological tissue section (or the fixed biological sample) in new (fresh) TBS-T-Modified at room temperature for two minutes.
5. The analysis procedure (for example, an immunohistochemistry procedure, an immunofluorescence procedure, an insitu hybridization and capture procedure, etc.) may be performed immediately on the fixed biological tissue section (or the fixed biological sample) following Step 4 of Protocol Six.
6. Alternatively, if desired following step 4 of Protocol Six, the fixed biological tissue section (or the fixed biological sample) may be held in the TBS-T-Modified at room temperature for up to 48 hours at a TBS-T-Modified temperature of 4° C. to 8° C. prior to initiating the desired analysis procedure (for example, an immunohistochemistry procedure, an immunofluorescence procedure, an insitu hybridization and capture procedure, etc.)

### Analysis of Fixed Tissue Sections

Immunohistochemistry Protocol Seven:

1. If the fixed biological tissue section (or fixed biological sample) was held in Tris-buffered saline-Tween, Modified (TBS-T-Modified) at a temperature of 4° C. to 8° C. prior to starting this Protocol, then first repeat Step 4 of Fixation Protocol Six before initiating Step 2 of this protocol.
2. Immerse the fixed tissue section obtained in Fixation Protocol Six in an aqueous room temperature solution containing 3 volume percent H₂O₂ (hydrogen peroxide) for ten minutes to block endogenous peroxidase activity. The H₂O₂ should be no more than three weeks old and should be stored at a temperature of 2° C. to 8° C. when not being used. Newcomer Supply of Middleton, Wis. is a suitable source of 30 volume percent H₂O₂ in water that may diluted with water to make 3 volume percent H₂O₂ in water.
3. Rinse the blocked fixed tissue section obtained in Step 2 three times with room temperature TBS-T-Modified, and then blot the perimeter of the tissue section, without actually touching the tissue section, to remove residual hydrogen peroxide solution.
4. Apply room temperature Fc receptor block to the tissue section, and allow ten minutes of incubation before again blotting the perimeter of the tissue section, without actually touching the tissue section, to remove residual Fc receptor block solution. Suitable Fc receptor block may be obtained from Innovex Biosciences of Richmond, Calif.
5. Apply the primary antibody in sufficient quantity to fully cover all exposed surfaces of the tissue section; incubate the tissue section overnight with the applied primary antibody at a temperature of 2° C. to 4° C. (or at the working incubation time and temperature recommended by the primary antibody supplier depending on the primary antibody dilution). Numerous examples of primary antibodies suitable for use in this Immunohistochemistry Protocol Seven are provided earlier in the body of this document.
6. Rinse residual primary antibody from the tissue section using two separate two minute washes of room temperature TBS-T-Modified and proceed to Step 7 or directly to Step 8, as desired.
7. Following Step 6, the rinsed tissue section may be held overnight at a temperature of 2° C. to 4° C. prior to proceeding with Step 8 of this Protocol Seven, if desired. If the rinsed tissue section is held overnight at a temperature of 2° C. to 4° C., the rinsed tissue section should be rinsed the following morning once with a two minute wash of room temperature TBS-T-Modified and then be allowed to rest at room temperature for about 45 minutes before proceeding with Step 8 of this protocol.
8. Blot the perimeter of the tissue section, without actually touching the tissue section, to remove residual TBS-T-Modified.
9. Apply the secondary antibody in sufficient quantity to fully cover all exposed surfaces of the tissue section; incubate the tissue section with the applied secondary antibody at room temperature for about thirty minutes (or at the working incubation time and temperature recommended by the secondary antibody supplier depending on the secondary antibody dilution). Numerous examples of secondary antibodies suitable for use in this Immunohistochemistry Protocol Seven are provided earlier in the body of this document.
10. Rinse residual secondary antibody from the tissue section using three separate two minute washes of room temperature TBS-T-Modified, and then blot the perimeter of the tissue section, without actually touching the tissue section, to remove any remaining residual TBS-T-Modified.
11. Apply either (1) streptavidin-horse radish peroxidase (“HRP”) enzymatic label (for a Peroxidase System where the biological sample is rich in alkaline phosphatase) or (2) streptavidin-alkaline phosphatase (“AP”) enzymatic label (for an Alkaline Phosphatase System where the biological sample is rich in peroxidase) at the working incubation time, temperature, and dilution recommended by the HRP or AP supplier to label any secondary antibody attached to the primary antibody attached to antigen. Both streptavidin-horse radish peroxidase and streptavidin-alkaline phosphatase may be obtained from Biogenex Laboratories of San Ramon, Calif., from Innovex BioSciences of Richmond, Calif., and from Biocare Medical of Walnut, Calif. For biological samples, such as liver tissue, that are rich in alkaline phosphatase, the HRP enzymatic label is ordinarily selected, since the AP enzymatic label would non-specifically react with the alkaline phosphatase present in the sample and cause undesirable non-specific staining. For biological samples, such as kidney tissue, that are rich in peroxidase, the AP enzymatic label is ordinarily selected, since the HRP enzymatic label would non-specifically react with the peroxidase present in the sample and cause undesirable non-specific staining. When the biological sample is neither particularly rich in peroxidase nor alkaline phosphatase, the HRP enzymatic label is often selected since the DAB (diaminobenzidine) chromogen-generating dye used in combination with the HRP enzymatic label (see Step 13 below) tends to remain stable and viable longer under typical storage conditions than many chromogen-generating dyes that are used in combination with the HRP enzymatic label AP enzymatic label.
12. Rinse residual HRP or AP labeling enzyme from the tissue section using three separate two minute washes of room temperature TBS-T-Modified, and then blot the perimeter of the tissue section, without actually touching the tissue section, to remove any remaining residual labeling enzyme.
13. If the streptavidin-horse radish peroxidase (“HRP”) enzymatic label was employed in Step 11, apply room temperature DAB (diaminobenzidine) as the chromogen-generating dye to the tissue section for one to five minutes. The DAB will cause positive signals of the primary antibody attached to antigens of the tissue section to exhibit a brown color. A suitable example of DAB is the DAB product (Catalog No. NB 314 SBD) that may be obtained from Innovex BioSciences of Richmond, Calif. If the streptavidin-alkaline phosphatase (“AP”) enzymatic label was employed in Step 11, apply either (1) room temperature Nuclear Fast Red chromogen (available from Biogenex Laboratories of San Ramon, Calif., from Innovex BioSciences of Richmond, Calif., and from Biocare Medical of Walnut, Calif.) or (2) room temperature Vulcan Fast Red chromogen (available from Biocare Medical of Walnut, Calif.) to the tissue section for five to ten minutes. The Nuclear Fast Red chromogen and the Vulcan Fast Red chromogen will cause positive signals of the primary antibody attached to antigens of the tissue section to exhibit a bright pink to red color.
14. While the color is developing following application of the DAB, Nuclear Fast Red chromogen, or Vulcan Fast Red chromogen in accordance with Step 13 above, examine the tissue section under a microscope and stop the color development when the desired quantity and intensity of color development has occurred by rinsing the tissue section with room temperature water. An example of a suitable microscope is the Olympus BHT microscope (at 10× magnification) that is available from the U.S. office of Olympus Corporation of Melville, N.Y.
15. After rinsing the tissue section per Step 14 to stop color development, counterstain the tissue section with five to ten dips (actual number of dips dependent on contrast desired for the counterstain) in a hematoxylin solution and thereafter rinse in cold tap water to remove unreacted hematoxylin. Suitable examples of hematoxylin solutions are available under any of Order Nos. 1180A, 1180D, 1180G, 1201, 12013A, or 1202A from Newcomer Supply, Inc. of Middleton, Wis. Next, proceed to either Step 16 or Step 17, as appropriate. The hematoxylin present in the hematoxylin solution serves as a counterstain that stains negative or non-reactive portions of the tissue section so the negative portions of the tissue exhibit a blue to purple color.
16. For a Peroxidase System where the streptavidin-horse radish peroxidase (“HRP”) enzymatic label was employed in Step 11, dehydrate the counterstained tissue section by dipping twenty times in each of the following solutions in the sequence shown:
   i. Aqueous solution of 50 volume percent ethanol, then
   ii. Aqueous solution of 75 volume percent ethanol, then
   iii. Aqueous solution of 95 volume percent ethanol, then
   iv. ˜100 volume percent ethanol (balance water), then
   v. ˜100 volume percent ethanol (balance water), then
   vi. 100 volume percent xylene, then
   vii. 100 volume percent xylene.

After the last dip in xylene, proceed to Step 18.
17. For an Alkaline Phosphatase System where the streptavidin-alkaline phosphatase (“AP”) enzymatic label was employed in Step 11, dehydrate the counterstained tissue section by dipping twenty times in each of the following solutions in the sequence shown:
   i. Aqueous solution of 95 volume percent ethanol, then
   ii. Aqueous solution of 95 volume percent ethanol, then
   iii. Aqueous solution of 95 volume percent ethanol, then
   iv. ˜100 volume percent ethanol (balance water), then
   v. ˜100 volume percent ethanol (balance water), then
   vi. ˜100 volume percent ethanol (balance water). After the last dip in xylene, proceed to Step 19.
18. For a Peroxidase System where the streptavidin-horse radish peroxidase (“HRP”) enzymatic label was employed in Step 11, mount a coverslip on the counterstained and dehydrated tissue section using PERMOUNT® mounting medium or an equivalent xylene-soluble mounting medium. PERMOUNT® mounting medium may be obtained from Fisher Scientific, International of Hampton, N.H. After coverslip mounting, proceed to Step 20.
19. For an Alkaline Phosphatase System where the streptavidin-alkaline phosphatase (“AP”) enzymatic label was employed in Step 11, mount a coverslip on the counterstained and dehydrated tissue section using the SUPERMOUNT® mounting medium available from Biogenex Laboratories of San Ramon, Calif., the ADVANTAGE™ permanent mounting medium available from Innovex Biosciences of Richmond, Calif., or an equivalent aqueous mounting medium. After coverslip mounting, proceed to Step 20.
20. Examine each coverslipped tissue section for reactivity (positive staining) under a microscope. An example of a suitable microscope is the Olympus BHT microscope (at 20× magnification) that is available from the U.S. office of Olympus Corporation of Melville, N.Y.
21. Following tissue section examination, store the examined slide in conditions appropriate for any desired future use or reexamination.
   Immofluorescence Protocol Eight:(For Confocal Microscope or Fluorescence Microscopy):
1. Rinse the slide containing the fixed biological tissue section (or fixed biological sample) in new (fresh) room temperature Tris-buffered saline-Tween, Modified (TBS-T-Modified) twice for three minutes (each rinse).
2. Immerse the rinsed fixed tissue section in room temperature aqueous solution containing 3 volume percent H₂O₂ (hydrogen peroxide) for ten minutes to block endogenous peroxidase activity. The H₂O₂ should be no more than three weeks old and should be stored at a temperature of 2° C. to 8° C. when not being used. Newcomer Supply of Middleton, Wis. is suitable source of 30 volume percent H₂O₂ in water that may be diluted with water to make 3 volume percent H₂O₂ in water.
3. Rinse the blocked fixed tissue section obtained in Step 2 three times with room temperature TBS-T-Modified, and then blot the perimeter of the tissue section, without actually touching the tissue, to remove residual hydrogen peroxide solution.
4. Perform an avidin biotin block on any fixed tissue section that is rich in biotin using room temperature avidin biotin block solution per the instructions provided with the particular avidin biotin block kit employed. One suitable avidin biotin block-kit is the Avidin/biotin blocking kit (Catalog No. HK 102-10K) that is available from Biogenex Laboratories of San Ramon, Calif. After performing the avidin biotin block, blot the perimeter of the tissue section, without actually touching the tissue section, to remove residual avidin biotin block solution.
5. If step 4 of this Protocol Eight was performed, rinse the fixed tissue section one time for about two minutes with room temperature TBS-T-Modified to remove any residual avidin biotin block solution. Then, blot the perimeter of the tissue section, without actually touching the tissue section, to remove residual TBS-T-Modified.
6. Apply room temperature Fc receptor block to the tissue section, and allow ten minutes of incubation before again blotting the perimeter of the tissue section, without actually touching the tissue section, to remove residual Fc receptor block solution. Suitable Fc receptor block may be obtained from Innovex Biosciences of Richmond, Calif.
7. Apply the primary antibody in sufficient quantity to fully cover all exposed surfaces of the tissue section; incubate the tissue section overnight with the applied primary antibody at a temperature of 2° C. to 4° C. (or at the working incubation time and temperature recommended by the primary antibody supplier(s) depending on the primary antibody dilution). The dilution and incubation time of the primary antibody is preferably optimized by trial and error to minimize autofluorescence when viewing the complete tissue section through the microscope. Numerous examples of primary antibodies suitable for use in this Immunofluorescence Protocol Eight are provided earlier in the body of this document.
8. Rinse residual primary antibody from the tissue section using two separate two minute washes of room temperature Tris-buffered saline-Tween, Modified (TBS-T-Modified).
9. Following Step 8, the rinsed tissue section may be held overnight at a temperature of 2° C. to 4° C. prior to proceeding with Step 10 of this Protocol Eight, if desired. If the rinsed tissue section are held overnight at a temperature of 2° C. to 4° C., the rinsed tissue section should be rinsed the following morning once with a two minute wash of room temperature TBS-Modified and then be allowed to rest at room temperature for about 45 minutes before proceeding with Step 10 of this protocol.
10. Blot the perimeter of the tissue section, without actually touching the tissue section, to remove residual TBS-Modified.
11. Apply the secondary antibody in sufficient quantity to fully cover all exposed surfaces of the tissue section; incubate the tissue section with the applied secondary antibody at room temperature for about thirty minutes (or at the working incubation time and temperature recommended by the secondary antibody supplier depending on the secondary antibody dilution). Numerous examples of secondary antibodies suitable for use in this Immunofluorescence Protocol Eight are provided earlier in the body of this document.
12. Rinse residual secondary antibody from the tissue section using three separate two minute washes of room temperature TBS-T-Modified, and then blot the perimeter of the tissue section, without actually touching the tissue section, to remove any remaining residual secondary antibody.
13. Apply streptavidin-horse radish peroxidase enzymatic label (“HRP”) at the working incubation time, temperature, and dilution recommended by the HRP supplier to label any secondary antibody attached to the primary antibody attached to antigen within the tissue section. Streptavidin-horse radish peroxidase may be obtained from Biogenex Laboratories of San Ramon, Calif., from Innovex BioSciences of Richmond, Calif., and from Biocare Medical of Walnut, Calif.
14. Rinse residual HRP labeling enzyme from the tissue section using three separate five minute washes of room temperature TBS-Modified, and then blot the perimeter of the tissue section, without actually touching the tissue section, to remove any remaining residual HRP labeling enzyme.

### Note: Steps 15 Through 17 Below should Occur in Darkness or Under a Dark Cloth

15. Apply a suitable signal detection solution, such as a room temperature solution of Tyramide Rhodamine (Red) fluorochrome or Tyramide Fluorescein (green) fluorochrome, to the tissue section at a weight ratio of 1:25 to 1:150 (weight ratio of Tyramide agent to aqueous diluent) for five to ten minutes or at the working incubation time and dilution recommended by a suitable signal detection solution supplier. The incubation time and dilution ratio (in aqueous diluent) will typically need to be optimized by trial and error observations depending on the availability of target antigens and the concentration of the primary antibody (in aqueous diluent) employed in Step 7 of this Protocol Eight. Tyramide Rhodamine (Red) may be obtained using Catalog No. NEL702 and Tyramide Fluorescein (green) may be obtained using Catalog No. NEL701 from Life Science Products, Inc. of Boston, Mass.
16. After application of the signal detection solution(s) per Step 15, rinse the tissue section three times for three minutes per rinse using distilled water.
17. After rinsing the tissue section per Step 16, counterstain the tissue section by incubating the tissue section with a dilute room temperature solution of DAPI (4,6-diamidino-2-phenylindole) for about five to about eight minutes. The dilute DAPI solution may be prepared by mixing one milligram of DAPI (powdered) in one liter of a phosphate buffer solution with a pH of about 7. Suitable powdered DAPI may be obtained from Merck KGaA of Darmstadt, Germany.
18. After application of the DAPI counterstaining solution, rinse the tissue section three times for three minutes per rinse using distilled water.
19. Next, mount a coverslip on the counterstained tissue section using SlowFade™ antifade mounting medium or an equivalent antifade mounting medium. SlowFade™ antifade mounting medium may be obtained from Molecular Probes, Inc. of Eugene, Oreg. After mounting the coverslip, proceed to Step 20.
20. Examine the coverslipped tissue section for fluorescent reactivity (positive staining) using a suitable confocal microscope or a suitable fluorescence microscope, as desired. Suitable examples of confocal microscopes include any of the confocal microscopes available from Leica Microsystems, Inc. of Bannockburn, Ill. Likewise, suitable examples of fluorescence microscopes include any of the fluorescence microscopes available from Leica Microsystems, Inc. It is generally suitable to examine the coverslipped tissue section under magnifications of 20× to 40×, though use of magnifications lower than 20× and use of magnifications greater than 40× may be desirable for particular applications.
21. Following tissue section examination, store the examined slide in conditions appropriate for any desired future use or reexamination.
   Immunofluorescence Protocol Nine:
   Double Labeling for Confocal Microscopy or Fluorescence Microscopy:
1. Perform Steps 1 through 5 of Immunofluorescence Protocol Eight and then proceed to Step 2 below.
2. Apply the first primary antibody in sufficient quantity to fully cover all exposed surfaces of the tissue section; incubate the tissue section overnight with the applied first primary antibody at a temperature of 2° C. to 4° C. (or at the working incubation time and temperature recommended by the first primary antibody supplier(s) depending on the first primary antibody dilution). The dilution and incubation time of the first primary antibody is preferably optimized by trial and error to minimize autofluorescence when viewing the complete tissue section through the microscope. Numerous examples of primary antibodies suitable for use in this Immunofluorescence Protocol Nine are provided earlier in the body of this document.
3. Rinse residual first primary antibody from the tissue section using two separate two minute washes of room temperature Tris-buffered saline-Tween, Modified (TBS-T-Modified).
4. Following Step 3, the rinsed tissue section may be held overnight at a temperature of 2° C. to 4° C. prior to proceeding with Step 5 of this Protocol Nine, if desired. If the rinsed tissue section is held overnight at a temperature of 2° C. to 4° C., the rinsed tissue section should be rinsed the following morning once with a two minute wash of room temperature TBS-Modified and then be allowed to rest at room temperature for about 45 minutes before proceeding with Step 5 of this Protocol.
5. Blot the perimeter of the tissue section, without actually touching the tissue section, to remove residual TBS-Modified.
6. Apply the first secondary antibody that is compatible with the first primary antibody in sufficient quantity to fully cover all exposed surfaces of the tissue section; incubate the tissue section with the applied first secondary antibody at room temperature for about thirty minutes (or at the working incubation time and temperature recommended by the first secondary antibody supplier depending on the first secondary antibody dilution). Numerous examples of secondary antibodies suitable for use in this Immunofluorescence Protocol Nine are provided earlier in the body of this document.
7. Rinse residual first secondary antibody from the tissue section using three separate two minute washes of room temperature TBS-T-Modified, and then blot the perimeter of the tissue section, without actually touching the tissue section, to remove any remaining residual first secondary antibody.
8. Apply streptavidin-horse radish peroxidase enzymatic label (“HRP”) at the working incubation time, temperature, and dilution recommended by the HRP supplier to label any first secondary antibody attached to the first primary antibody attached to antigen within the tissue section. Streptavidin-horse radish peroxidase may be obtained from Biogenex Laboratories of San Ramon, Calif., from Innovex BioSciences of Richmond, Calif., and from Biocare Medical of Walnut, Calif.
9. Rinse residual HRP labeling enzyme from the tissue section using three separate five minute washes of room temperature TBS-Modified, and then blot the perimeter of the tissue section, without actually touching the tissue section, to remove any remaining residual HRP labeling enzyme.

### Note: Steps 10 Through 25 Below should Occur in Darkness or Under a Dark Cloth

10. Apply a suitable signal detection solution, such as a room temperature solution of Tyramide Rhodamine (Red) fluorochrome to the tissue section at a weight ratio of 1:25 to 1:150 (weight ratio of Tyramide agent to aqueous diluent) for five to ten minutes or at the working incubation time and dilution recommended by a suitable signal detection solution supplier. The incubation time and dilution ratio (in aqueous diluent) will typically need to be optimized by trial and error observations depending on the availability of target antigens and the concentration of the first primary antibody (in aqueous diluent) employed in step 2 of this Protocol Nine. Tyramide Rhodamine (Red) may be obtained using Catalog No. NEL702 from Life Science Products, Inc. of Boston, Mass.
11. After application of the signal detection solution per Step 10, rinse the tissue section three times for three minutes per rinse using distilled water.
12. Rinse the tissue section following Step 11 three times with room temperature TBS-T-Modified, and then blot the perimeter of the tissue section, without actually touching the tissue, to remove any remaining residual Tyramide Rhodamine (Red) fluorochrome.
13. Apply room temperature Fc receptor block to the tissue section, and allow ten minutes of incubation before again blotting the perimeter of the tissue section, without actually touching the tissue section, to remove residual Fc receptor block solution. Suitable Fc receptor block may be obtained from Innovex Biosciences of Richmond, Calif.
14. Apply the second primary antibody in sufficient quantity to fully cover all exposed surfaces of the tissue section; incubate the tissue section overnight with the applied second primary antibody at a temperature of 2° C. to 4° C. (or at the working incubation time and temperature recommended by the second primary antibody supplier(s) depending on the second primary antibody dilution). The dilution and incubation time of the second primary antibody is preferably optimized by trial and error to minimize autofluorescence when viewing the complete tissue section through the microscope. The animal source of the second primary antibody may optionally be different from the animal source of the first primary antibody.
15. Rinse residual second primary antibody from the tissue section using two separate two minute washes of room temperature Tris-buffered saline, modified (TBS-Modified).
16. Following Step 15, the rinsed tissue section may be held overnight at a temperature of 2° C. to 4° C. prior to proceeding with Step 17 of this Protocol Nine, if desired. If the rinsed tissue section is held overnight at a temperature of 2° C. to 4° C., the rinsed tissue section should be rinsed the following morning once with a two minute wash of room temperature TBS-Modified and then be allowed to rest at room temperature for about 45 minutes before proceeding with Step 17 of this Protocol.
17. Blot the perimeter of the tissue section, without actually touching the tissue section, to remove residual TBS-Modified.
18. Apply the second secondary antibody that is compatible with the second primary antibody in sufficient quantity to fully cover all exposed surfaces of the tissue section; incubate the tissue section with the applied second secondary antibody at room temperature for about thirty minutes (or at the working incubation time and temperature recommended by the second secondary antibody supplier depending on the second secondary antibody dilution).
19. Rinse residual second secondary antibody from the tissue section using three separate two minute washes of room temperature TBS-Modified, and then blot the perimeter of the tissue section, without actually touching the tissue section, to remove any remaining residual second secondary antibody.
20. Apply streptavidin-horse radish peroxidase enzymatic label (“HRP”) at the working incubation time, temperature, and dilution recommended by the HRP supplier to label any second secondary antibody attached to the second primary antibody attached to antigen within the tissue section. Streptavidin-horse radish peroxidase may be obtained from Biogenex Laboratories of San Ramon, Calif., from Innovex BioSciences of Richmond, Calif., and from Biocare Medical of Walnut, Calif.
21. Rinse residual HRP labeling enzyme from the tissue section using three separate five minute washes of room temperature TBS-Modified, and then blot the perimeter of the tissue section, without actually touching the tissue section, to remove any remaining residual HRP labeling enzyme.
22. Apply a suitable signal detection solution, such as a room temperature solution of Tyramide Fluorescein (green) fluorochrome to the tissue section at a weight ratio of 1:25 to 1:150 (weight ratio of Tyramide agent to aqueous diluent) for five to ten minutes or at the working incubation time and dilution recommended by a suitable signal detection solution supplier. The incubation time and dilution ratio (in aqueous diluent) will typically need to be optimized by trial and error observations depending on the availability of target antigens and the concentration of the second primary antibody (in aqueous diluent) employed in step 14 of this Protocol Nine. Tyramide Fluorescein (green) may be obtained using Catalog No. NEL701 from Life Science Products, Inc. of Boston, Mass.
23. After application of the signal detection solution per Step 22, rinse the tissue section three times for three minutes per rinse using distilled water.
24. After rinsing the tissue section per Step 23, counterstain the tissue section by incubating the tissue section with a dilute room temperature solution of DAPI (4,6-diamidino-2-phenylindole) for about five to about eight minutes. The dilute DAPI solution may be prepared by mixing one milligram of DAPI (powdered) in one liter of a phosphate buffer solution with a pH of about 7. Suitable powdered DAPI may be obtained from Merck KGaA of Darmstadt, Germany.
25. After application of the DAPI counterstaining solution, rinse the tissue section three times for three minutes per rinse using distilled water.
26. Next, mount a coverslip on the counterstained tissue section using SlowFade™ antifade mounting medium or an equivalent antifade mounting medium. SlowFade™ antifade mounting medium may be obtained from Molecular Probes, Inc. of Eugene, Oreg. After mounting the coverslip, proceed to Step 27.
27. Examine the coverslipped tissue section for fluorescent reactivity (positive staining) using a suitable confocal microscope or a suitable fluorescence microscope, as desired. See Step 20 of Immunofluorescence Protocol Eight for some microscope and magnification suggestions.
28. Following tissue section examination, store the examined slide in conditions appropriate for any desired future use or reexamination.
   Hematoxylin And Eosin Staining Protocol Ten:
1. Rinse the slide containing the fixed biological tissue section (or fixed biological sample) in new (fresh) room temperature Tris-buffered saline-Tween, Modified (TBS-T-Modified) by dipping the fixed biological tissue section in the TBS-T-Modified ten times.
2. Following Step 1, rinse the fixed tissue section under running room temperature tap water twenty times so the fixed tissue section is immersed in the tap water for a total of about sixty seconds.
3. Stain the fixed tissue section with hematoxylin by dipping the fixed tissue section in hematoxylin solution twenty times so the fixed tissue section is immersed in the hematoxylin solution for a total of about sixty seconds. The total number of dips in the hematoxylin solution, duration of immersion in the hematoxylin solution, and concentration of hematoxylin in the hematoxylin solution may be varied up or down as desired and appropriate for different analysis or evaluation procedures. Suitable examples of hematoxylin solutions are available under any of Order Nos. 1180A, 1180D, 1180G, 1201, 12013A, or 1202A from Newcomer Supply, Inc. of Middleton, Wis.
4. Rinse the fixed tissue section in running room temperature water twenty times so the fixed tissue section is immersed in the tap water for a total of about sixty seconds to stop color development and remove unreacted hematoxylin.
5. Examine the fixed tissue section under a microscope and confirm if sufficient hematoxylin staining has occurred to yield the desired color intensity. An example of a suitable microscope is the Olympus BHT microscope (at 10× magnification) that is available from the U.S. office of Olympus Corporation of Melville, N.Y.
6. If sufficient color intensity is observed in Step 5, proceed to Step 8 below. If insufficient color intensity is observe in Step 5, further stain the fixed tissue section with hematoxylin by dipping the fixed tissue section in the hematoxylin solution twenty more times so the fixed tissue section is immersed in the hematoxylin solution for a total of about sixty seconds.
7. Again rinse the fixed tissue section in running room temperature water twenty times so the fixed tissue section is immersed in the tap water for a total of about sixty seconds to remove unreacted hematoxylin.
8. Dip the hematoxylin-stained fixed tissue section in bluing solution twenty times so the fixed tissue section is immersed in the bluing solution for a total of about sixty seconds. The bluing solution helps develop the color following reaction of the hematoxylin with the tissue target. Suitable bluing solution is available from Newcomer Supply, Inc. of Middleton, Wis.
9. Again rinse the fixed tissue section in running room temperature water twenty times so the fixed tissue section is immersed in the tap water for a total of about sixty seconds to stop color development and remove unreacted bluing solution.
10. Following step 9, sequentially immerse the fixed tissue section in an aqueous ethanol solution (about 95 volume percent ethanol and about 5 volume percent water) twenty times so the fixed tissue section is immersed in the ethanol solution for a total of about sixty seconds.
11. Following step 10, counterstain the fixed tissue section in an aqueous Eosin solution for about twelve minutes. The duration of immersion in the Eosin solution and concentration of Eosin powder in the Eosin solution may be varied up or down as desired and appropriate for different analysis or evaluation procedures. Suitable examples of Eosin solutions are available under any of Order Nos. 1068B, 1070B, or 1080B from Newcomer Supply, Inc. of Middleton, Wis.
12. Following step 11, sequentially immerse the fixed tissue section in an aqueous ethanol solution (about 95 volume percent ethanol and about 5 volume percent water) twenty times so the fixed tissue section is immersed in the ethanol solution for a total of about sixty seconds.
13. Following step 12, sequentially immerse the fixed tissue section in another aqueous ethanol solution (about 99.8 volume percent or more ethanol, the balance water and denaturant) twenty times so the fixed tissue section is immersed in the ethanol solution for a total of about sixty seconds.
14. Following step 13, sequentially immerse the fixed tissue section in xylene twenty times so the fixed tissue section is immersed in the xylene for a total of about sixty seconds.
15. Next, mount a coverslip on the counterstained tissue section using Cytoseal™ mounting medium or an equivalent mounting medium. Cytoseal™ antifade mounting medium may be obtained from Electron Microscopy Sciences of Fort Washington, Pa. After mounting the coverslip, proceed to Step 16.
16. Examine each coverslipped tissue section for reactivity (positive staining) under a microscope. An example of a suitable microscope is the Olympus BHT microscope (at 20× magnification) that is available from the U.S. office of Olympus Corporation of Melville, N.Y.
17. Following tissue section examination, store the examined slide in conditions appropriate for any desired future use or reexamination.

### EXAMPLES AND COMPARATIVE EXAMPLES

In the various Comparative Examples provided below, formalin, ethanol, methanol, and acetone are employed as comparative fixatives for purposes of performance comparison to the FROZFIX® fixative of the present invention that is employed in Examples 1-9. The formalin employed in the Comparative Examples was a neutral buffered ten volume percent formalin solution containing about ten volume percent of a commercially available aqueous formaldehyde solution (containing about 37 to about 40 volume percent formaldehyde, about 11 to about 14 volume percent methanol, and water to make 100 volume percent) and water to make 100 volume percent of the neutral buffered ten volume percent formalin solution. The ethanol employed in the Comparative Examples was a commercially-obtained research grade of ethanol containing about 95 volume percent, or more, ethanol, about 2 volume percent, or less, of a denaturant (such as methanol), and the balance water. The methanol employed in the Comparative Examples was a commercially-obtained technical grade of methanol containing about 95 volume percent, or more, methanol and the balance water. The acetone employed in the Comparative Examples was a reagent grade of acetone containing about 99 volume percent, or more, acetone, about 1.0 volume percent, or less, water, and the balance minor amounts of other polar solvents.

For each particular Example and each group of Comparative Examples related to the particular Example, both positive and negative controls were provided to confirm the reliability of the observed results. The positive and negative controls used in the various Examples and Comparative Examples were subjected to the same Protocols listed below for the particular Examples and Comparative Examples, unless otherwise indicated. The positive and negative controls used in the various Examples and Comparative Examples consisted of the same biological sample described for each particular Example and each particular Comparative Example.

### Example 1 and Comparative Examples 1A-1D

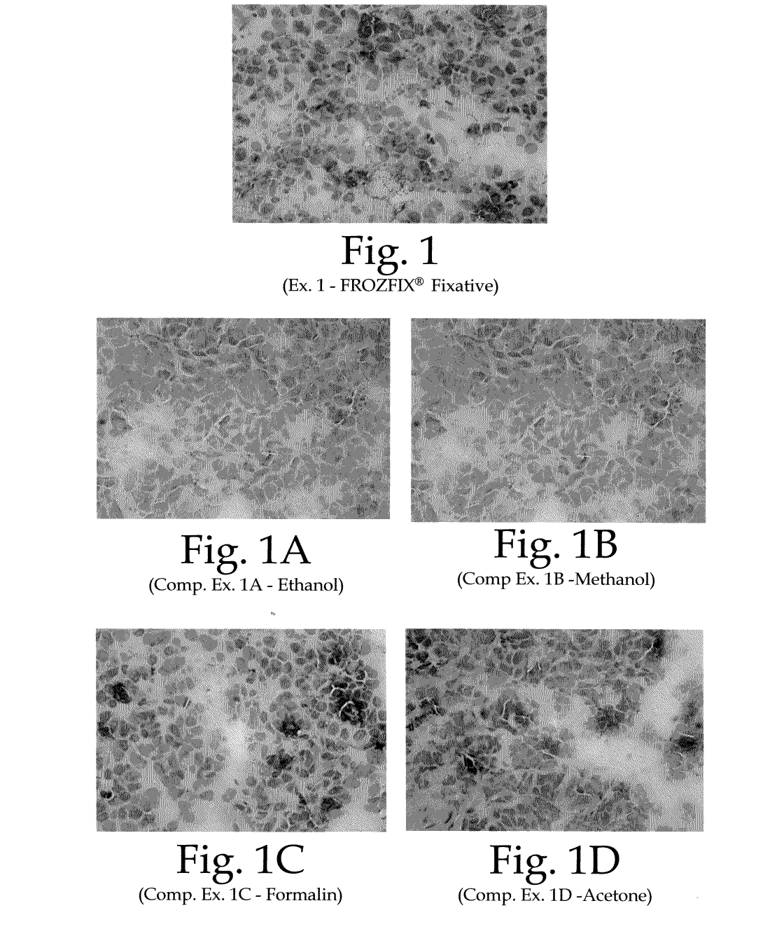

In Example 1, Protocol One and Protocols Four through Seven were followed using FROZFIX® fixative, which is a biological fixative produced in accordance with the present invention. The FROZFIX® fixative was incorporated in a previously-frozen human tonsil tissue section as described in Fixation Protocol Six. The primary antibody employed in Example 1 was the FCR 5 antibody (source: mouse); a rabbit anti-mouse antibody was employed as the secondary antibody in Example 1. The tonsil tissue used in Example 1 utilized a Peroxidase System; therefore, streptavidin-horse radish peroxidase (“HRP”) was used as the enzymatic label in Example 1. Due to the detection system employed in Example 1, the location of the antigen and attached primary antibody (FCR 5) is indicated by a brown color. A micrograph visually depicting immuno localization of the antibody FCR 5 in human tonsil tissue fixed using the FROZFIX® fixative of the present invention applied in accordance with the fixation method of the present invention is depicted in FIG. 1.

In Comparative Examples 1A-1D, Protocol One and Protocols Four through Seven were generally followed with the exception that the FROZFIX® fixative was not employed in any of Comparative Examples 1A-1D. Instead, the fixatives used in Comparative Examples 1A-1D were ethanol, methanol, formalin, and acetone, respectively. The same primary antibody employed in Example 1 was also employed in Comparative Examples 1A-1D; likewise, the same secondary antibody employed in Example 1 was also employed in Comparative Examples 1A-1D. Due to the detection system employed in Comparative Examples 1A-1D, the location of the antigen and attached primary antibody (FCR 5) is indicated by a brown color. Micrographs attempting to visually depict immuno localization of the antibody FCR 5 in human tonsil tissue fixed in accordance with Comparative Examples 1A-1D are depicted in FIGS. 1A-1D, respectively.

In Example 1 and in Comparative Examples 1A-1D, normal human lymph node tissue known to be positive for the FCR 5 antibody employed in Example 1 and in Comparative Examples 1A-1D was employed as a positive control. For the positive controls for each of Example 1 and Comparative Examples 1A-1D, all steps of Protocol Seven were performed.

Also, for Example 1 and for each of Comparative Examples 1A-1D, control tissue known to be negative for the FCR 5 antibody employed in Example 1 and in Comparative Examples 1A-1D was provided and evaluated. For the negative controls for each of Example 1 and Comparative Examples 1A-1D, all steps of Protocol Seven were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-horse radish peroxidase (“HRP”) enzymatic label, but not the DAB (diaminobenzidine) chromogen-generating dye, was applied to the tissue sample; and (4) in a fourth negative control, the DAB (diaminobenzidine) chromogen-generating dye, but not the streptavidin-horse radish peroxidase (“HRP”) enzymatic label, was applied to the tissue sample.

From FIGS. 1A-1B, it is evident the ethanol and methanol used in Comparative Examples 1A and 1B resulted in negative staining (no brown-colored signal—therefore no location of the antigen is shown and no indication is given of any primary antibody attached to the antigen). While the formalin and acetone employed in Comparative Examples 1C and 1D showed some positive staining, the significant voids between counterstained (blue) areas indicate significant morphology compromise occurred, as seen in FIGS. 1C-1D. On the other hand, the FROZFIX® fixative of the present invention employed in Example 1 (see FIG. 1) illustrates improved morphology stabilization compared to at least the acetone along with better antigenicity stabilization relative to the acetone and the formalin (and the ethanol and methanol).

### Example 2 and Comparative Examples 2A-2B

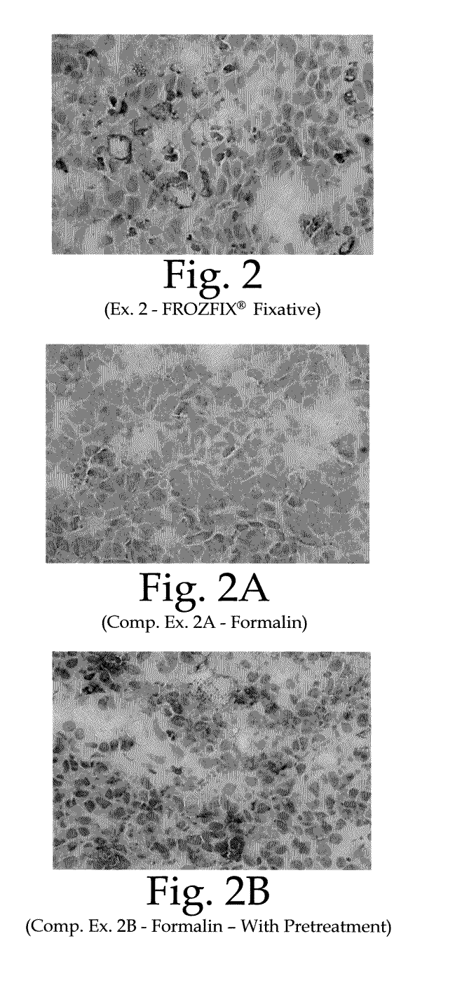

In Example 2, Protocol One and Protocols Four through Seven were followed using FROZFIX® fixative produced in accordance with the present invention. The FROZFIX® fixative was incorporated in a previously-frozen human tonsil tissue section as described in Fixation Protocol Six. The primary antibody employed in Example 2 was the FCR 1 antibody (source: mouse); a rabbit anti-mouse antibody was employed as the secondary antibody in Example 2. The tonsil tissue used in Example 2 utilized a Peroxidase System; therefore, streptavidin-horseradish peroxidase (“HRP”) was used as the enzymatic label in Example 2. Due to the detection system employed in Example 2, the location of the antigen and attached primary antibody (FCR 1) is indicated by a brown color. A micrograph visually depicting immuno localization of the antibody FCR 1 in human tonsil tissue fixed using the FROZFIX® fixative of the present invention applied in accordance with the fixation method of the present invention is depicted in FIG. 2.

In Comparative Examples 2A-2B, Protocol One and Protocols Four through Seven were generally followed with the exception that the FROZFIX® fixative was not employed in Comparative Examples 2A-2B. Instead, the fixative used in both Comparative Examples 2A and 2B was formalin. In Comparative Example 2A, no enzymatic pretreatment occurred prior to initiating Protocol Seven; in Comparative Example 2B, enzymatic pretreatment employing Trypsin was undertaken prior to initiating Protocol Seven. The same primary antibody employed in Example 2 was also employed in Comparative Examples 2A and 2B; likewise, the same secondary antibody employed in Example 2 was also employed in Comparative Examples 2A and 2B. Due to the detection system employed in Comparative Examples 2A-2B, the location of the antigen and attached primary antibody (FCR 1) is indicated by a brown color. Micrographs attempting to visually depict immuno localization of the antibody FCR 1 in human tonsil tissue fixed in accordance with Comparative Examples 2A-2B are depicted in FIGS. 2A and 2B, respectively.

In Example 2 and in Comparative Examples 2A-2B, normal human lymph node tissue known to be positive for the FCR 1 antibody employed in Example 2 and in Comparative Examples 2A-2B was employed as a positive control. For the positive controls for each of Example 2 and in Comparative Examples 2A-2B, all steps of Protocol Seven were performed.

Also, for Example 2 and for each of Comparative Examples 2A-2B, control tissue known to be negative for the FCR 1 antibody employed in Example 2 and in Comparative Examples 2A-2B was provided and evaluated. For the negative controls for each of Example 2 and Comparative Examples 2A-2B, all steps of Protocol Seven were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-horse radish peroxidase (“HRP”) enzymatic label, but not the DAB (diaminobenzidine) chromogen-generating dye, was applied to the tissue sample; and (4) in a fourth negative control, the DAB (diaminobenzidine) chromogen-generating dye, but not the streptavidin-horse radish peroxidase (“HRP”) enzymatic label, was applied to the tissue sample.

From FIG. 2, it is evident the FROZFIX® fixative of the present invention employed in Example 2 resulted in positive staining (strong signal indicating the location of the antigen and attached primary antibody), whereas the formalin employed in Comparative Example 2A (see FIG. 2A) resulted in negative staining (no brown-colored signal—therefore no location of the antigen is shown and no indication is given of any primary antibody attached to the antigen). While the formalin and enzymatic pretreatment employed in Comparative Example 2B (see FIG. 2B) did show positive staining, it only occurred by employing the extra enzymatic pretreatment step. No such pretreatment step was required in Example 2 to attain the beneficial results when using the FROZFIX® fixative of the present invention. Use of the FROZFIX® fixative of the present invention thus beneficially illustrates good morphology stabilization along with better antigenicity stabilization relative to formalin.

### Example 3 and Comparative Examples 3A-3D

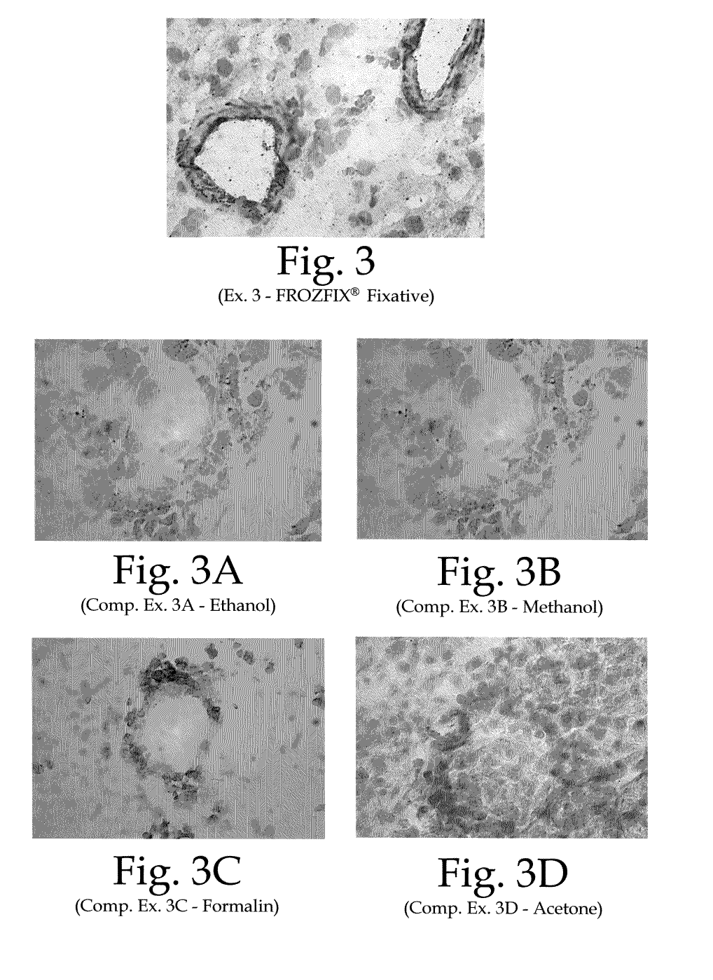

In Example 3, Protocol One and Protocols Four through Seven were followed using FROZFIX® fixative, which is a biological fixative produced in accordance with the present invention. The FROZFIX® fixative was incorporated in a previously-frozen human brain tumor section as described in Fixation Protocol Six. The primary antibody employed in Example 3 was the protein Actin (source: mouse); a rabbit anti-mouse antibody was employed as the secondary antibody in Example 3. The human brain tumor used in Example 3 utilized an Alkaline Peroxidase System; therefore, streptavidin-alkaline phosphatase (“AP”) was used as the enzymatic label in Example 3. Due to the detection system employed in Example 3, the location of the antigen and attached primary antibody (Actin) is indicated by a red color. A micrograph visually depicting immuno localization of the primary antibody (Actin) in human brain tumor fixed using the FROZFIX® fixative of the present invention applied in accordance with the fixation method of the present invention is depicted in FIG. 3.

In Comparative Examples 3A-3D, Protocol One and Protocols Four through Seven were generally followed with the exception that the FROZFIX® fixative was not employed in any of Comparative Examples 3A-3D. Instead, the fixatives used in Comparative Examples 3A-3D were ethanol, methanol, formalin, and acetone, respectively. The same primary antibody employed in Example 3 was also employed in Comparative Examples 3A-3D; likewise, the same secondary antibody employed in Example 3 was also employed in Comparative Examples 3A-3D. Due to the detection system employed in Comparative Examples 3A-3D, the location of the antigen and attached primary antibody (Actin) is indicated by a red color. Micrographs attempting to visually depict immuno localization of the primary antibody (Actin) in human brain tumor fixed in accordance with Comparative Examples 3A-3D are depicted in FIGS. 3A-3D, respectively.

In Example 3 and in Comparative Examples 3A-3D, normal human brain tissue known to be positive for the primary antibody (Actin) employed in Example 3 and in Comparative Examples 3A-3D was employed as a positive control. For the positive controls for each of Example 3 and Comparative Examples 3A-3D, all steps of Protocol Seven were performed.

Also, for Example 3 and for each of Comparative Examples 3A-3D, control tissue known to be negative for the primary antibody (Actin) employed in Example 3 and in Comparative Examples 3A-3D was provided and evaluated. For the negative controls for each of Example 3 and Comparative Examples 3A-3D, all steps of Protocol Seven were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-alkaline phosphatase (“AP”) enzymatic label, but not the Vulcan Fast Red chromogen-generating dye, was applied to the tissue sample; and (4) in a fourth negative control, the Vulcan Fast Red chromogen-generating dye, but not the streptavidin-alkaline phosphatase (“AP”) enzymatic label, was applied to the tissue sample.

From FIGS. 3A-3B, it is evident the ethanol and methanol used in Comparative Examples 3A and 3B resulted in negative staining (no red-colored signal—therefore no location of the antigen is shown and no indication is given of any primary antibody attached to the antigen). While the formalin employed in Comparative Example 3C (see FIG. 3C) showed some positive staining, the staining is weak and diffuse; this is apparently due to the strong crosslinking effect of the formalin with the consequent effect that antigens, though likely present, are predominantly unavailable for primary antibody attachment. While the acetone employed in Comparative Example 3D (see FIG. 3D) showed a significant amount of positive staining, the diffuse nature of the red color indicates the acetone use substantially compromised the morphology of the human brain tumor. On the other hand, the FROZFIX® fixative of the present invention employed in Example 3 (see FIG. 3) illustrates improved morphology stabilization (more and crisper red staining) compared to the acetone along with much better antigenicity stabilization relative to the formalin (and the ethanol and methanol).

### Example 4 and Comparative Examples 4A-4E

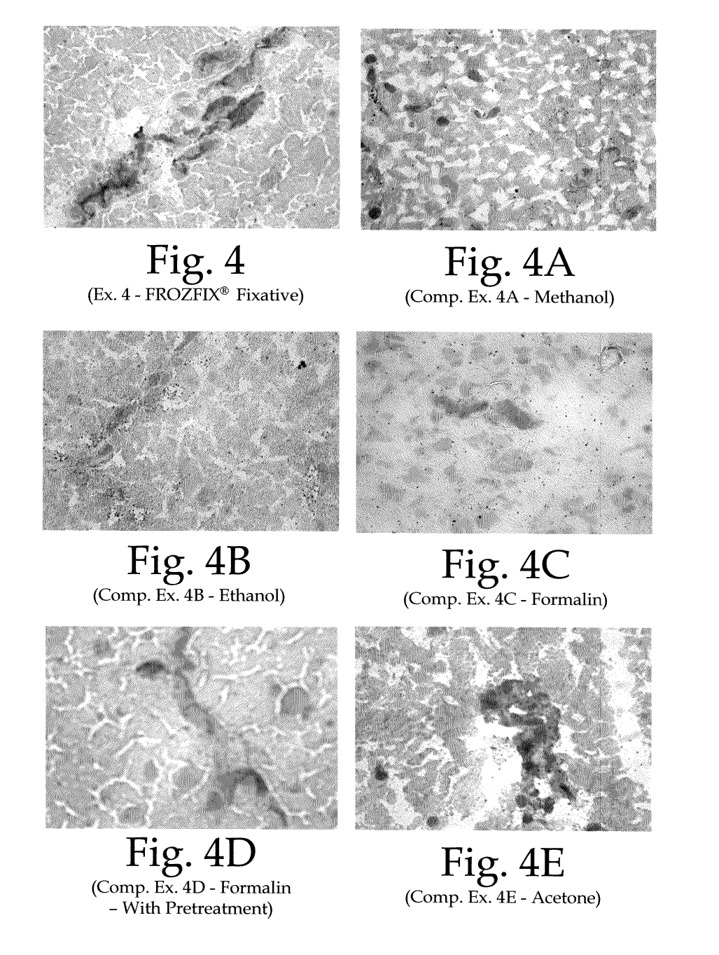

In Example 4, Protocol One and Protocols Four through Seven were followed using FROZFIX® fixative, which is a biological fixative produced in accordance with the present invention. The FROZFIX® fixative was incorporated in a previously-frozen normal human brain tissue section as described in Fixation Protocol Six. The primary antibody employed in Example 4 was the protein Actin (source: mouse); a rabbit anti-mouse antibody was employed as the secondary antibody in Example 4. The normal human brain tissue used in Example 4 utilized an Alkaline Peroxidase System; therefore, streptavidin-alkaline phosphatase (“AP”) was used as the enzymatic label in Example 4. Due to the detection system employed in Example 4, the location of the antigen and attached primary antibody (Actin) is indicated by a red color. A micrograph visually depicting immuno localization of the primary antibody (Actin) in normal human brain tissue fixed using the FROZFIX® fixative of the present invention applied in accordance with the fixation method of the present invention is depicted in FIG. 4.

In Comparative Examples 4A-4E, Protocol One and Protocols Four through Seven were generally followed with the exception that the FROZFIX® fixative was not employed in any of Comparative Examples 4A-4E. Instead, the fixatives used in Comparative Examples 4A-4C and 4E were methanol, ethanol, formalin, and acetone, respectively. Formalin was also used as the fixative in Comparative Example 4D. In Comparative Example 4C, no enzymatic pretreatment occurred prior to initiating Protocol Seven; in Comparative Example 4D, enzymatic pretreatment was undertaken prior to initiating Protocol Seven. The same primary antibody employed in Example 4 was also employed in Comparative Examples 4A-4E; likewise, the same secondary antibody employed in Example 4 was also employed in Comparative Examples 4A-4E. Due to the detection system employed in Comparative Examples 4A-4E, the location of the antigen and attached primary antibody (Actin) is indicated by a red color. Micrographs attempting to visually depict immuno localization of the primary antibody (Actin) in normal human brain tissue fixed in accordance with Comparative Examples 4A-4E are depicted in FIGS. 4A-4E, respectively.

In Example 4 and in Comparative Examples 4A-4E, normal human skin tissue known to be positive for the primary antibody (Actin) employed in Example 4 and in Comparative Examples 4A-4E was employed as a positive control. For the positive controls for each of Example 4 and Comparative Examples 4A-4E, all steps of Protocol Seven were performed.

Also, for Example 4 and for each of Comparative Examples 4A-4E, control tissue known to be negative for the primary antibody (Actin) employed in Example 4 and in Comparative Examples 4A-4E was provided and evaluated. For the negative controls for each of Example 4 and Comparative Examples 4A-4E, all steps of Protocol Seven were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-alkaline phosphatase (“AP”) enzymatic label, but not the Vulcan Fast Red chromogen-generating dye, was applied to the tissue sample; and (4) in a fourth negative control, the Vulcan Fast Red chromogen-generating dye, but not the streptavidin-alkaline phosphatase (“AP”) enzymatic label, was applied to the tissue sample.

From FIGS. 4A-4B, it is evident the methanol and ethanol used in Comparative Examples 4A and 4B resulted in negative staining (no red-colored signal—therefore no location of the antigen is shown and no indication is given of any primary antibody attached to the antigen). While the formalin employed in Comparative Example 4C (see FIG. 4C) showed some positive staining, the staining is weak and diffuse; this is apparently due to the strong crosslinking effect of the formalin with the consequent effect that antigens, though likely present, are predominantly unavailable for antibody attachment. While the acetone employed in Comparative Example 4E (see FIG. 4E) showed a significant amount of positive staining, the diffuse nature of the red color indicates the acetone use substantially compromised the morphology of the normal human brain tissue.

While the formalin and enzymatic pretreatment employed in Comparative Example 4D (see FIG. 4D) did show positive staining, it only occurred by employing the extra enzymatic pretreatment step; also, the diffuse nature of the red color indicates the formalin and enzymatic pretreatment approach significantly compromised the morphology of the normal human brain tissue. On the other hand, the FROZFIX® fixative of the present invention employed in Example 4 (see FIG. 4) illustrates improved morphology stabilization (more and crisper red staining) compared to the acetone and the formalin with enzymatic pretreatment along with much better antigenicity stabilization relative to the formalin alone (and the ethanol and methanol). Furthermore, no pretreatment step was required in Example 4 to attain the beneficial results when using the FROZFIX® fixative of the present invention.

### Example 5 and Comparative Examples 5A-5D

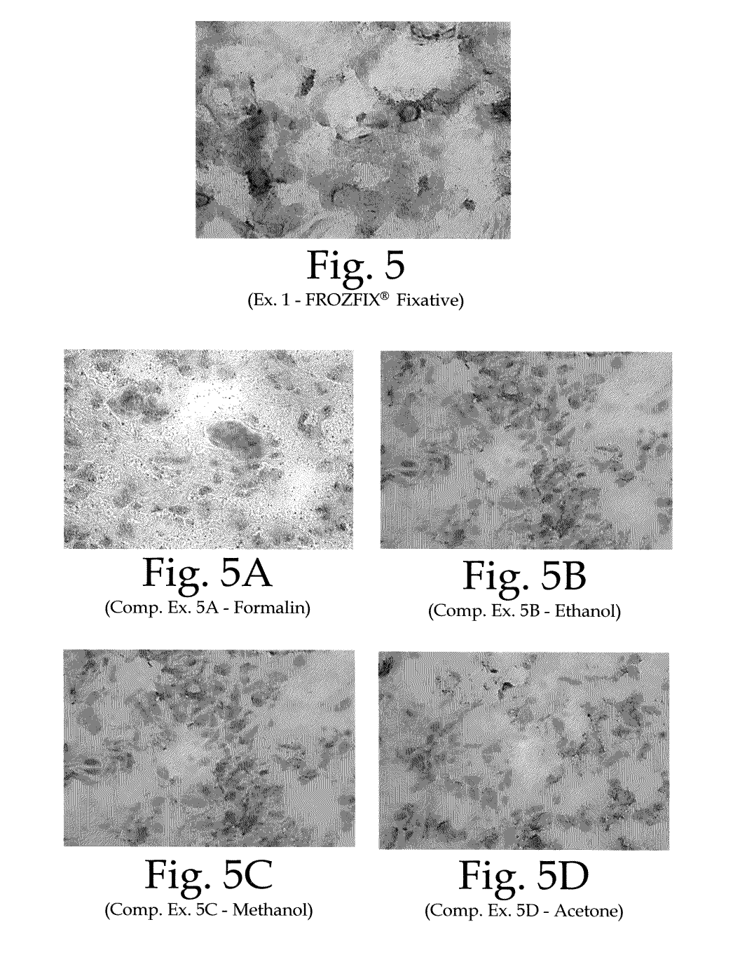

In Example 5, Protocol One and Protocols Four through Seven were followed using FROZFIX® fixative, which is a biological fixative produced in accordance with the present invention. The FROZFIX® fixative was incorporated in a previously-frozen human brain tumor section as described in Fixation Protocol Six. The primary antibody employed in Example 5 was Cytomegalovirus (source: mouse); a rabbit anti-mouse antibody was employed as the secondary antibody in Example 5. The human brain tumor used in Example 5 utilized an Alkaline Peroxidase System; therefore, streptavidin-alkaline phosphatase (“AP”) was used as the enzymatic label in Example 5. Due to the detection system employed in Example 5, the location of the antigen and attached primary antibody (Cytomegalovirus) is indicated by a red color. A micrograph visually depicting immuno localization of the primary antibody (Cytomegalovirus) in the human brain tumor fixed using the FROZFIX® fixative of the present invention applied in accordance with the fixation method of the present invention is depicted in FIG. 5.

In Comparative Examples 5A-5D, Protocol One and Protocols Four through Seven were generally followed with the exception that the FROZFIX® fixative was not employed in any of Comparative Examples 5A-5D. Instead, the fixatives used in Comparative Examples 5A-5D were formalin, ethanol, methanol, and acetone, respectively. The same primary antibody employed in Example 5 was also employed in Comparative Examples 5A-5D; likewise, the same secondary antibody employed in Example 5 was also employed in Comparative Examples 5A-5D. Due to the detection system employed in Comparative Examples 5A-5D, the location of the antigen and attached primary antibody (Cytomegalovirus) is indicated by a red color. Micrographs attempting to visually depict immuno localization of the primary antibody (Cytomegalovirus) in human brain tumor fixed in accordance with Comparative Examples 5A-5D are depicted in FIGS. 5A-5D, respectively.

In Example 5 and in Comparative Examples 5A-5D, human lung tissue known to be positive for (infected with) the primary antibody (Cytomegalovirus) employed in Example 5 and in Comparative Examples 5A-5D was employed as a positive control. For the positive controls for each of Example 5 and Comparative Examples 5A-5D, all steps of Protocol Seven were performed.

Also, for Example 5 and for each of Comparative Examples 5A-5D, control tissue known to be negative for the primary antibody (Cytomegalovirus) employed in Example 5 and in Comparative Examples 5A-5D was provided and evaluated. For the negative controls for each of Example 5 and Comparative Examples 5A-5D, all steps of Protocol Seven were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-alkaline phosphatase (“AP”) enzymatic label, but not the Vulcan Fast Red chromogen-generating dye, was applied to the tissue sample; and (4) in a fourth negative control, the Vulcan Fast Red chromogen-generating dye, but not the streptavidin-alkaline phosphatase (“AP”) enzymatic label, was applied to the tissue sample.

From FIG. 5A, it is evident the formalin used in Comparative Example 5A resulted in negative staining (no red-colored signal—therefore no location of the antigen is shown and no indication is given of any primary antibody attached to the antigen). While the ethanol, methanol, and acetone employed in Comparative Examples 5B-5D (see FIGS. 5B-5D) showed more positive staining than the formalin of Comparative Example 5A, the diffuse nature of the red color indicates the ethanol, methanol, and acetone use substantially compromised the morphology of the human brain tumor. On the other hand, the FROZFIX® fixative of the present invention employed in Example 5 (see FIG. 5) illustrates improved morphology stabilization (crisper red staining) compared to the ethanol, methanol, and acetone along with better antigenicity stabilization relative to the ethanol, methanol, and acetone (and the formalin).

### Example 6 and Comparative Examples 6A-6D

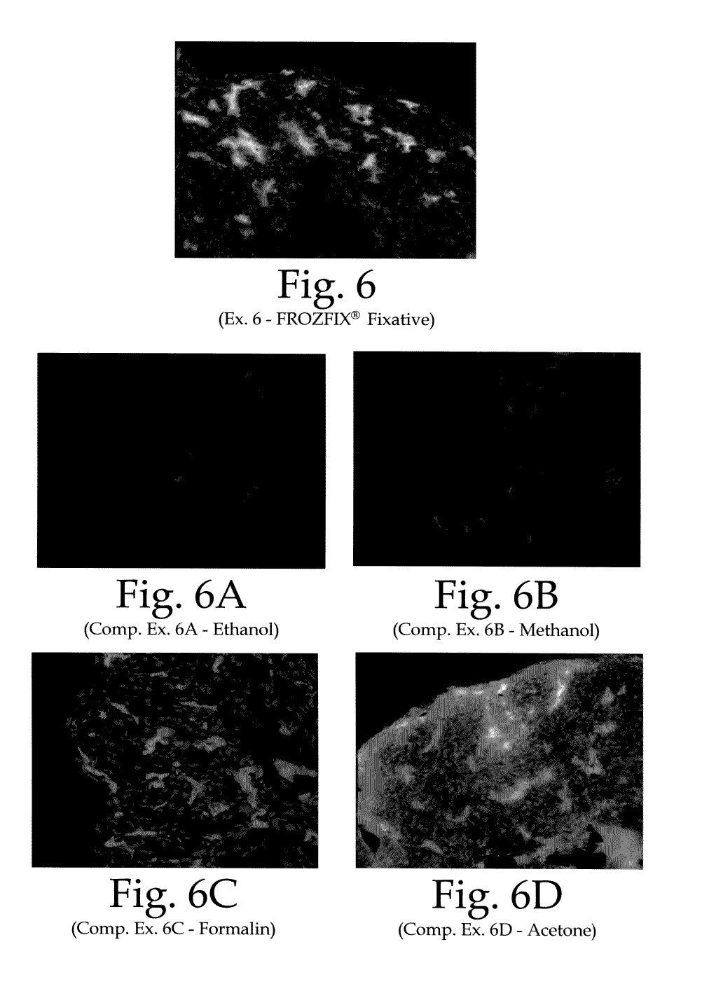

In Example 6, Protocols One, Four through Six, and Eight were followed using FROZFIX® fixative, which is a biological fixative produced in accordance with the present invention. The immunofluorescent observations made in Protocol Eight were performed using confocal microscopy. The FROZFIX® fixative was incorporated in previously-frozen murine epithelial tissue section as described in Fixation Protocol Six. The primary antibody employed in Example 6 was MHC Class II antibody (source: mouse); a rabbit anti-mouse antibody was employed as the secondary antibody in Example 6. Due to the Tyramide Rhodamine (Red) detection system employed in Example 6, the location of the antigen and attached primary antibody (MHC Class II antibody) is indicated by a red color. A micrograph visually depicting labeling of the primary antibody (MHC Class II antibody) in the murine epithelial tissue fixed using the FROZFIX® fixative of the present invention applied in accordance with the fixation method of the present invention is depicted in FIG. 6.

In Comparative Examples 6A-6D, Protocols One, Four through Six, and Eight were generally followed with the exception that the FROZFIX® fixative was not employed in any of Comparative Examples 6A-6D. Instead, the fixatives used in Comparative Examples 6A-6D were ethanol, methanol, formalin, and acetone, respectively. The same primary antibody employed in Example 6 was also employed in Comparative Examples 6A-6D; likewise, the same secondary antibody employed in Example 6 was also employed in Comparative Examples 6A-6D. Due to the detection system employed in Comparative Examples 6A-6D, the location of the antigen and attached primary antibody (MHC Class II antibody) is indicated by a red color. Micrographs attempting to visually depict labeling of the primary antibody (MHC Class II antibody) in murine epithelial tissue fixed in accordance with Comparative Examples 6A-6D are depicted in FIGS. 6A-6D, respectively.

In Example 6 and in Comparative Examples 6A-6D, rat pouch (cheek) tissue known to be positive for the MHC Class II antibody employed in Example 6 and in Comparative Examples 6A-6D was employed as a positive control. For the positive controls for each of Example 6 and in Comparative Examples 6A-6D, all steps of Protocol Eight were performed.

Also, for Example 6 and for each of Comparative Examples 6A-6D, control tissue known to be negative for the MHC Class II antibody employed in Example 6 and in Comparative Examples 6A-6D was provided and evaluated. For the negative controls for each of Example 6 and Comparative Examples 6A-6D, all steps of Protocol Eight were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-horse radish peroxidase (“HRP”) enzymatic label, but not the Tyramide Rhodamine (Red) fluorochrome, was applied to the tissue sample; and (4) in a fourth negative control, the Tyramide Rhodamine (Red) fluorochrome, but not the streptavidin-horse radish peroxidase (“HRP”) enzymatic label, was applied to the tissue sample.

From FIGS. 6A-6B, it is evident the ethanol and methanol used in Comparative Example 6A and 6B resulted in negative staining (no red-colored signal—therefore no location of the antigen is shown and no indication is given of any primary antibody attached to the antigen). While the formalin used in Comparative Example 6C (see FIG. 6C) showed more positive staining than the ethanol and methanol used in Comparative Examples 6A and 6B, the staining is weak and diffuse; this is apparently due to the strong crosslinking effect of the formalin with the consequent effect that antigens, though likely present, are predominantly unavailable for antibody attachment. Though the acetone of Comparative Example 6D (see FIG. 6D) shows strong staining, the staining is highly diffused and non-specific. The diffuse and non-specific nature of the red color indicates the acetone use substantially compromised the morphology of the murine epithelial tissue; the compromised morphology is believed due to the dehydrating effect of the acetone. On the other hand, the FROZFIX® fixative of the present invention employed in Example 6 (see FIG. 6) illustrates improved morphology stabilization (crisper red staining) compared to the ethanol, methanol, and acetone along with better antigenicity stabilization relative to the formalin (and the ethanol and methanol).

### Example 7 and Comparative Examples 7A-7D

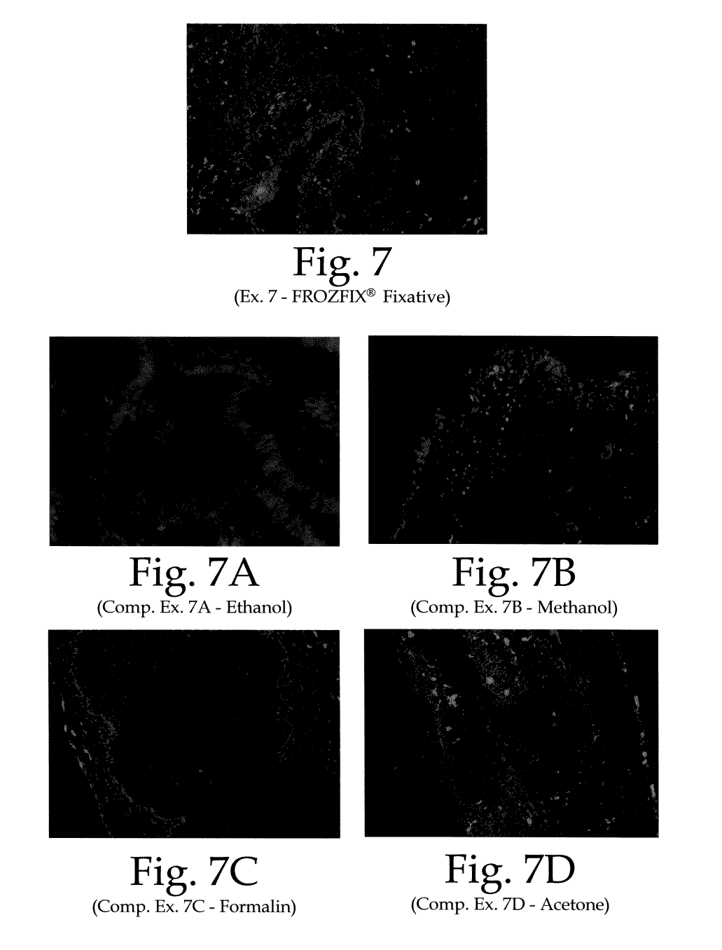

In Example 7, Protocols One, Four through Six, and Eight were followed using FROZFIX® fixative, which is a biological fixative produced in accordance with the present invention. The immunofluorescent observations made in Protocol Eight were obtained using fluorescent microscopy. The FROZFIX® fixative was incorporated in a previously-frozen murine epithelial tissue section as described in Fixation Protocol Six. The primary antibody employed in Example 7 was antibody CD11c (source: mouse); a rabbit anti-mouse antibody was employed as the secondary antibody in Example 7. Due to the Tyramide Rhodamine (Red) detection system employed in Example 7, the location of the antigen and attached primary antibody (CD11c) is indicated by a red color. A micrograph visually depicting labeling of the primary antibody (CD11c) in the murine epithelial tissue fixed using the FROZFIX® fixative of the present invention applied in accordance with the fixation method of the present invention is depicted in FIG. 7.

In Comparative Examples 7A-7D, Protocols One, Four through Six, and Eight were generally followed with the exception that the FROZFIX® fixative was not employed in any of Comparative Examples 7A-7D. Instead, the fixatives used in Comparative Examples 7A-7D were ethanol, methanol, formalin, and acetone, respectively. The same primary antibody employed in Example 7 was also employed in Comparative Examples 7A-7D; likewise, the same secondary antibody employed in Example 7 was also employed in Comparative Examples 7A-7D. Due to the detection system employed in Comparative Examples 7A-7D, the location of the antigen and attached primary antibody (CD11c) is indicated by a red color. Micrographs attempting to visually depict labeling of the primary antibody (CD11c) in murine epithelial tissue fixed in accordance with Comparative Examples 7A-7D are depicted in FIGS. 7A-7D, respectively.

In Example 7 and in Comparative Examples 7A-7D, rat pouch (cheek) tissue known to be positive for the CD11c antibody employed in Example 7 and in Comparative Examples 7A-7D was employed as a positive control. For the positive controls for each of Example 7 and in Comparative Examples 7A-7D, all steps of Protocol Eight were performed.

Also, for Example 7 and for each of Comparative Examples 7A-7D, control tissue known to be negative for the CD11c antibody employed in Example 7 and in Comparative Examples 7A-7D was provided and evaluated. For the negative controls for each of Example 7 and Comparative Examples 7A-7D, all steps of Protocol Eight were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-horse radish peroxidase (“HRP”) enzymatic label, but not the Tyramide Rhodamine (Red) fluorochrome, was applied to the tissue sample; and (4) in a fourth negative control, the Tyramide Rhodamine (Red) fluorochrome, but not the streptavidin-horse radish peroxidase (“HRP”) enzymatic label, was applied to the tissue sample.

From FIGS. 7A-7B, it is evident the ethanol used in Comparative Example 7A resulted in negative staining (no red-colored signal—therefore no location of the antigen is shown and no indication is given of any primary antibody attached to the antigen), while the methanol used in Comparative Example 7B resulted in very weak staining. While the formalin used in Comparative Example 7C (see FIG. 7C) showed more positive staining than the ethanol and methanol used in Comparative Examples 7A and 7B, the staining is weak and fairly diffuse; this is apparently due to the strong crosslinking effect of the formalin with the consequent effect that antigens, though likely present, are predominantly unavailable for antibody attachment. Though the acetone of Comparative Example 7D (see FIG. 7D) shows strong staining, the staining is substantially non-specific. The non-specific nature of the red color indicates the acetone use significantly compromised the morphology of the murine epithelial tissue. On the other hand, the FROZFIX® fixative of the present invention employed in Example 7 (see FIG. 7) illustrates improved morphology stabilization (crisper red staining) compared to the ethanol, methanol, and acetone along with better antigenicity stabilization relative to the formalin (and the ethanol and methanol).

### Example 8 and Comparative Examples 8A-8D

In Example 8, Protocols One, Six, and Nine were followed using FROZFIX® fixative, which is a biological fixative produced in accordance with the present invention. The immunofluorescent observations made in Protocol Eight were obtained using fluorescence microscopy. The infected tissue culture was grown directly on an examination slide that was included as part of a chamber slide; after growing the culture, the chamber was removed to leave the examination slide containing the infected tissue culture. Since the grown culture existed as a monolayer on the examination slide, there was no need to section the culture or apply frozen processing media to the culture. Therefore, Protocols Four and Five were skipped in Example 8. After the tissue culture was frozen per Protocol One, the frozen tissue culture was immersed in the FROZFIX® fixative per Step 1 of Fixation Protocol Six and thereafter the remainder of Protocol Six was followed.

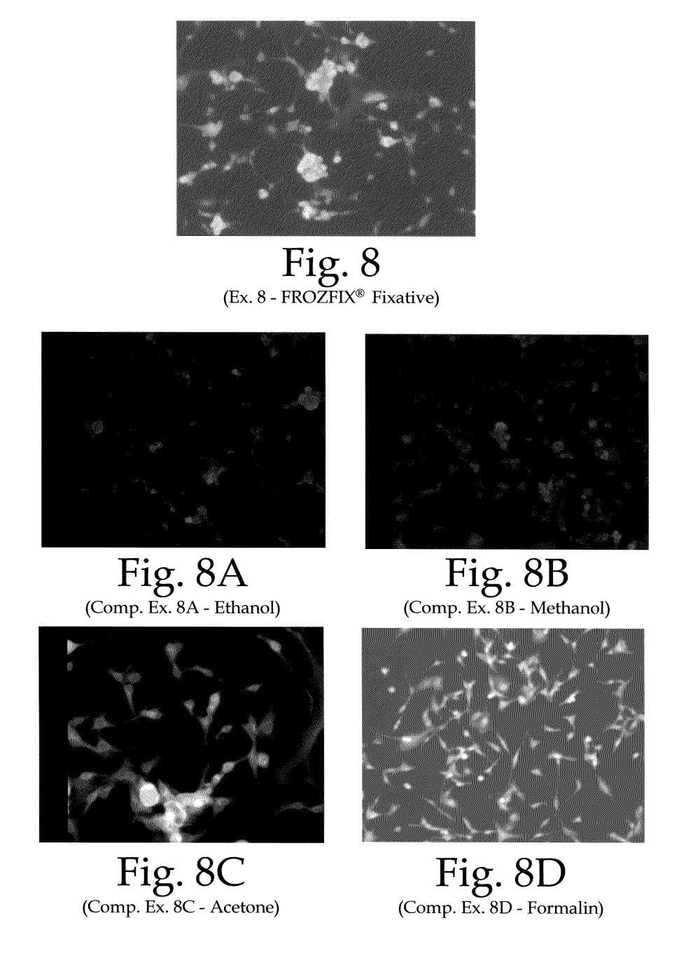

Double labeling with two different primary antibodies and two different secondary corresponding, respectively, to the two different primary antibodies was undertaken in Example 8. The first primary antibody employed in Example 8 was Cytomegalovirus (source: mouse), which is intended to bind to (engage with) a first antigen present in the tissue culture. The second primary antibody employed in Example 8 was NFK-β antibody (source: rabbit), which is intended to bind to (engage with) a second antigen present in the tissue culture. A rabbit anti-mouse antibody was employed as the first secondary antibody compatible with the first primary antibody in Example 8. A goat anti-rabbit antibody was employed as the second secondary antibody compatible with the second primary antibody in Example 8. A micrograph visually depicting labeling of (1) the first antigen and the first primary antibody (Cytomegalovirus) engaged with the first antigen in the infected tissue culture and (2) the second antigen and the second primary antibody (NFK-β antibody) engaged with the second antigen in the infected tissue culture, where the infected tissue culture has been fixed using the FROZFIX® fixative of the present invention applied in accordance with the fixation method of the present invention, is depicted in FIG. 8.

Due to the detection system employed in Example 8, the location of the first antigen and any first primary antibody engaged with the first antigen in FIG. 8 is indicated by a light green color. Likewise, due to the detection system employed in Example 8, the location of the second antigen and any second primary antibody engaged with the second antigen is indicated in FIG. 8 by a reddish to pinkish color. If both (1) the first antigen (and any first primary antibody engaged with the first antigen) and (2) the second antigen (and any second primary antibody engaged with the second antigen) are co-localized in the same region of the tissue culture (so the first and second antigen share the same are of antigenicity) this co-localized area where the fist antigen and the second antigen are present is indicated in FIG. 8 by a yellow color. The yellow color is a result of the combination of (1) the light green color indicating the presence of the first antigen and engaged first primary antibody and (2) the red to pink color indicating the presence of the second antigen and engaged second primary antibody.

In Comparative Examples 8A-8D, Protocols One, Six, and Nine were generally followed with a couple of exceptions. First, the FROZFIX® fixative was not employed in any of Comparative Examples 8A-8D. Instead, the fixatives used in Comparative Examples 8A-8D were ethanol, methanol, acetone and formalin, respectively. Infected tissue culture was used as the biological sample in Comparative Examples 8A-8D, as in Example 8. The infected tissue culture was grown directly on a dish with perforated sections that were removed to leave an examination slide containing the infected tissue culture. Since the grown culture existed as a monolayer on the examination slide, there was no need to section the culture or apply frozen processing media to the culture. Therefore, Protocols Four and Five were skipped in Comparative Examples 8A-8D. A further exception is the frozen tissue culture created via Protocol One was immersed in the comparative fixative per Step 1 of Fixation Protocol Six, since the comparative fixative was substituted in place of the FROZFIX® fixative in Protocol Six for Comparative Examples 8A-8D. Thereafter, the remainder of Protocol Six was followed.

Double labeling was employed in Comparative Examples 8A-8D as in Example 8; the same two primary antibodies and the same two secondary antibodies employed in Example 8 were also employed in Comparative Examples 8A-8D. Due to the detection system employed in Example 8, the location of the first antigen and any attached first primary antibody is indicated by a light green color, and the location of the second antigen and any attached second primary antibody is indicated by a reddish to pinkish color. As in Example 8, areas where the first antigen (and attached first primary antibody) and the second antigen (an attached second primary antibody) are co-localized are indicated by a yellow color. Micrographs attempting to visually depict labeling of (1) the first antigen and the first primary antibody (Cytomegalovirus) engaged with the first antigen in the infected tissue culture and (2) the second antigen and the second primary antibody (NFK-β antibody) engaged with the second antigen in the infected tissue culture fixed in accordance with Comparative Examples 8A-8D are depicted in FIGS. 8A-8D, respectively.

In Example 8 and in Comparative Examples 8A-8D, a culture of human brain tumor cells (cell Line U251) known to be positive for both the Cytomegalovirus antibody (AD 169 strain) and for the NFK-β antibody employed in Example 8 and in Comparative Examples 8A-8D was employed as a positive control. For the positive controls for each of Example 8 and in Comparative Examples 8A-8D, all steps of Protocol Nine were performed.

Also, for Example 8 and for each of Comparative Examples 8A-8D, a culture of human brain tumor cells (cell Line U251) known to be positive for both the Cytomegalovirus antibody (AD 169 strain) and for the NFK-β antibody employed in Example 8 and in Comparative Examples 8A-8D was provided and evaluated. For the negative controls for each of Example 8 and Comparative Examples 8A-8D, all steps of Protocol Nine were performed, except as indicated for the six different negative controls below: (1) in a first negative control, none of the first primary antibody, the second primary antibody, the first secondary antibody, or the second secondary antibody was applied to the tissue culture; (2) in a second negative control, the first secondary antibody, the second primary antibody, the second primary antibody, but not any of the first primary antibody, was applied to the tissue culture; (3) in a third negative control, the first secondary antibody, the first primary antibody, the second primary antibody, but not any of the second secondary antibody, was applied to the tissue culture; (4) in a fourth negative control, the streptavidin-horse radish peroxidase (“HRP”) enzymatic label, but not any of the Tyramide Rhodamine (Red) fluorochrome or any of the Tyramide Fluorescein (Green) fluorochrome, was applied to the tissue culture; (4) in a fifth negative control, the Tyramide Rhodamine (Red) fluorochrome, but not any of streptavidin-horse radish peroxidase (“HRP”) enzymatic label or any of the Tyramide Fluorescein (Green) fluorochrome, was applied to the tissue culture; and (6) in a sixth negative control, the Tyramide Fluorescein (Green) fluorochrome, but not any of streptavidin-horse radish peroxidase (“HRP”) enzymatic label or any of the Tyramide Rhodamine (Red) fluorochrome, was applied to the tissue culture.

From FIGS. 8A and 8B, it is evident the ethanol and methanol used in Comparative Examples 8A and 8B resulted in negative staining for the Cytomegalovirus antibody (no light green signal—therefore no location of the first antigen is shown and no indication is given of any first primary antibody attached to the first antigen) and positive staining for the NFK-β antibody (red colored signal indicates the location of the second antigen and attached second primary antibody). Furthermore, the lack of any yellow colored signal in FIGS. 8A and 8B demonstrates that neither the ethanol used in Comparative Example A nor the methanol used in Comparative Example B produced evidence of any co-localization of (1) the first antigen (and attached first primary antibody) and (2) the second antigen (and attached second primary antibody).

While the formalin used in Comparative Example 8D (see FIG. 8D) shows both some greenish staining and some very small amounts of yellow staining, the greenish staining is diffuse, while the yellow staining is minimal. Both of these effects are apparently due to the strong crosslinking effect of the formalin with the consequent effect that the first and second antigens, though likely present, are predominantly unavailable for attachment of the first primary antibody to the first antigen and for attachment of the second primary antibody to the second antigen in the infected tissue culture.

From FIG. 8C, it is seen the acetone used in Comparative Example 8C showed some positive staining for (1) the Cytomegalovirus antibody (light green colored signal indicates the location of the first antigen and attached first primary antibody) (2) the NFK-β antibody (red colored signal indicates the location of the second antigen and attached second primary antibody), and (3) co-localization (light yellow) of (a) the first antigen and attached first primary antibody and (b) the second antigen and attached second primary antibody. Thus, the acetone of Comparative Example 8C appears to have performed better in overall visualization of the first antigen, the second antigen, and co-localization of the first and second antigens than either the ethanol and methanol used in Comparative Example 8A and 8B or the formalin used in Comparative Example 8D. However, the staining of Comparative Example 8C is fairly diffuse and weak; which indicates the acetone use significantly compromised the antigenicity of the infected tissue.

On the other hand, the FROZFIX® fixative of the present invention employed in Example 8 (see FIG. 8) illustrates improved antigenicity stabilization (crisper yellow, green, and pink staining) compared to the acetone of Comparative Example 8C. Likewise, the FROZFIX® fixative of the present invention employed in Example 8 yielded a significant amount of positive stains (pink color) for the NFK-β antibody and for the co-localization (yellow color) of the first antigen and the second antigen while the formalin of Comparative Example 8D yielded essentially no positive stains (pink color) for the NFK-β antibody and only minimal positive staining (yellow color) for the co-localization of the first antigen and the second antigen. Also, while the FROZFIX® fixative of the present invention employed in Example 8 yielded a significant amount of positive stains (pink color) for the NFK-β antibody and for the co-localization (yellow color) of the first antigen and the second antigen, the ethanol and methanol of Comparative Examples 8A and 8B yielded no positive stains (pink color) for the NFK-β antibody and no positive stains (yellow color) for the co-localization of the first antigen and the second antigen. Consequently, it is evident the FROZFIX® fixative of the present invention employed in Example 8 illustrates improved antigenicity stabilization compared to the ethanol and methanol of Comparative Examples 8A and 8B, compared to the acetone of Comparative Example 8C, and compared to the formalin of Comparative Example 8D.

### Example 9 and Comparative Examples 9A-9D

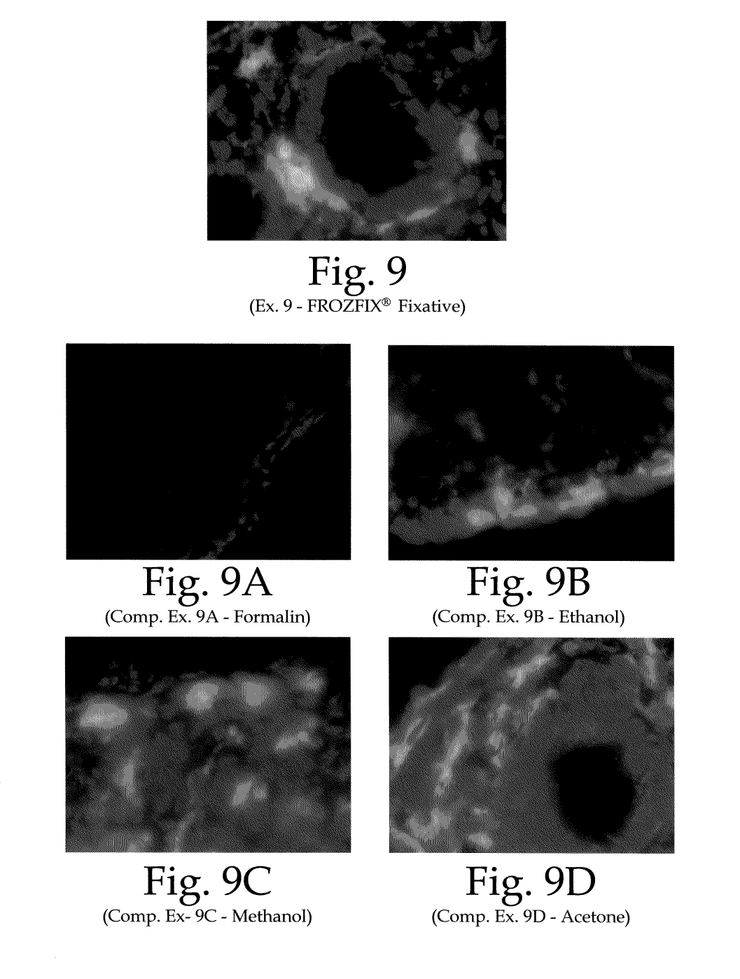

In Example 9, Protocols One, Four through Six, and Eight were followed using FROZFIX® fixative, which is a biological fixative produced in accordance with the present invention. The immunofluorescent observations made in Protocol Eight were performed using fluorescent microscopy. The FROZFIX® fixative was incorporated in a previously-frozen murine epithelial tissue section as described in Fixation Protocol Six. The primary antibody employed in Example 9 was MHC Class II antibody (source: rat). A hamster anti-rat antibody was employed as the secondary antibody in Example 9. Due to the Tyramide Rhodamine (Red) detection system employed in Example 9, the location of the antigen and attached primary antibody (MHC Class II antibody) is indicated by a red color. A micrograph visually depicting labeling of the primary antibody (MHC Class II antibody) in the murine epithelial tissue fixed using the FROZFIX® fixative of the present invention applied in accordance with the fixation method of the present invention is depicted in FIG. 9.

In Comparative Examples 9A-9D, Protocols One, Four through Six, and Eight were generally followed with the exception that the FROZFIX® fixative was not employed in any of Comparative Examples 9A-9D. Instead, the fixatives used in Comparative Examples 9A-9D were formalin, ethanol, methanol, and acetone, respectively. The same primary antibody employed in Example 9 was also employed in Comparative Examples 9A-9D; likewise, the same secondary antibody employed in Example 9 was also employed in Comparative Examples 9A-9D. Due to the signal detection system employed in Comparative Examples 9A-9D, the location of the antigen and attached primary antibody (MHC Class II antibody) is indicated by a red color. Micrographs attempting to visually depict labeling of the primary antibody (MHC Class II antibody) in murine epithelial tissue fixed in accordance with Comparative Examples 9A-9D are depicted in FIGS. 9A-9D, respectively.

In Example 9 and in Comparative Examples 9A-9D, rat pouch (cheek) tissue known to be positive for the MHC Class II antibody employed in Example 9 and in Comparative Examples 9A-9D was employed as a positive control. For the positive controls for each of Example 9 and in Comparative Examples 9A-9D, all steps of Protocol Eight were performed.

Also, for Example 9 and for each of Comparative Examples 9A-9D, control tissue known to be negative for the MHC Class II antibody employed in Example 9 and in Comparative Examples 9A-9D was provided and evaluated. For the negative controls for each of Example 9 and Comparative Examples 9A-9D, all steps of Protocol Eight were performed, except as indicated for the four different negative controls below: (1) in a first negative control, no primary or secondary antibody was applied to the tissue sample; (2) in a second negative control, secondary, but not primary antibody, was applied to the tissue sample; (3) in a third negative control, the streptavidin-horse radish peroxidase (“HRP”) enzymatic label, but not the Tyramide Rhodamine (Red) fluorochrome, was applied to the tissue sample; and (4) in a fourth negative control, the Tyramide Rhodamine (Red) fluorochrome, but not the streptavidin-horse radish peroxidase (“HRP”) enzymatic label, was applied to the tissue sample.

From FIGS. 9A-9B, it is evident the formalin used in Comparative Example 9A resulted in predominantly negative staining (essentially no red signal—therefore essentially no location of the antigen and of primary antibody attached to the antigen is shown), while the ethanol used in Comparative Example 9B resulted in only weak staining. The almost non-existent staining when formalin was used is apparently due to the strong crosslinking effect of the formalin with the consequent effect that antigen, though likely present, is predominantly unavailable for antibody attachment. While the methanol used in Comparative Example 9C (see FIG. 9C) showed more positive (red) staining than the formalin and ethanol used in Comparative Examples 9A and 9B, the staining is very diffuse and weak, which indicates the methanol use significantly compromised antigenicity stabilization in the murine epithelial tissue. Though the acetone of Comparative Example 9D (see FIG. 9D) shows stronger positive (red) staining than the formalin, ethanol, or methanol, the positive staining is fairly weak and very diffuse. The weak nature of the red color indicates the acetone use significantly compromised antigenicity stabilization in the murine epithelial tissue. On the other hand, the FROZFIX® fixative of the present invention employed in Example 9 (see FIG. 9) illustrates improved better antigenicity stabilization (crisper and stronger red staining) compared to the formalin, ethanol, methanol, and acetone.

Although the present invention has been described with reference to preferred embodiments, workers skilled in the art will recognize that changes may be made in form and detail without departing from the spirit and scope of the invention.

1. A composition, the composition comprising:
an aldehyde;
alcohol; and
a ketone; the volumetric ratio of the alcohol to the ketone in the composition ranging from as low as about 0.8:1 to as high as about 4.5:1 and the volumetric ratio of the alcohol to the aldehyde in the composition ranging from as low as about 41.5:1 to as high as about 450:1.

2. The composition of claim 1 wherein the aldehyde comprises formaldehyde.

3. The composition of claim 1 wherein the alcohol comprises ethanol.

4. The composition of claim 1 wherein the ketone comprises acetone.

5. The composition of claim 1 wherein the composition is pH buffered.

6. The composition of claim 1 wherein the volumetric ratio of the alcohol to the ketone in the composition ranges from as low as about 1.5:1 to as high as about 2.1:1.

7. The composition of claim 1 wherein the volumetric ratio of the alcohol to the aldehyde in the composition ranges from as low as about 97:1 to as high as about 152:1.

8. The composition of claim 1 wherein the volumetric ratio of the alcohol to the ketone in the composition is about 1.8:1 and the volumetric ratio of the alcohol to the aldehyde in the composition is about 120:1.

9. The composition of claim 1 wherein the composition, upon application to a biological sample, is effective to stabilize the morphology and antigenicity of the biological sample.

10. A composition for fixing a biological sample, the composition comprising:
an aldehyde;
alcohol;
a ketone; and;
water, wherein:
the aldehyde has a concentration ranging from as low as about 0.1 volume percent to as high as about 0.6 volume percent, based upon the total volume of the aldehyde, alcohol, ketone, and water in the composition; and
the volumetric ratio of the alcohol to the ketone in the composition ranges from as low as about 0.8:1 to as high as about 4.5:1.

11. The composition of claim 10 wherein the concentration of ketone in the composition ranges from as low as about 10 volume percent to as high as about 30 volume percent, based on the total volume of the aldehyde, alcohol, ketone, and water in the composition.

12. The composition of claim 10 wherein the aldehyde comprises formaldehyde.

13. The composition of claim 10 wherein the alcohol comprises ethanol.

14. The composition of claim 10 wherein the ketone comprises acetone.

15. The composition of claim 10 wherein the composition is pH buffered.

16. The composition of claim 10 wherein the composition, upon application to the biological sample, is effective to stabilize the morphology and antigenicity of the biological sample.

17. The composition of claim 10 wherein the volumetric ratio of the alcohol to the ketone in the composition ranges from as low as about 1.5:1 to as high as about 2.1:1.

18. The composition of claim 10 wherein the volumetric ratio of the alcohol to the ketone in the composition is about 1.8:1.

19. The composition of claim 10 wherein the aldehyde has a concentration ranging from as low as about 0.25 volume percent to as high as about 0.35 volume percent, based on the total volume of the aldehyde, alcohol, ketone, and water in the composition.

20. A material, the material comprising:
a frozen biological composition; and
a fluid composition in intimate contact with the frozen biological composition, the fluid composition comprising:
ketone;
alcohol; and
water; and
aldehyde.

21. The material of claim 20 wherein the frozen biological composition comprises a frozen smear of the biological composition.

22. The material of claim 21 wherein the biological composition comprises a hematology specimen, a medical dialysis fluid, bronchial lavage, a secretion from a body organ or tissue, a scrape-collected or swab-collected substance from a tissue lining, gastric fluid, peritoneal fluid, pleural fluid, synovial fluid, spinal fluid; a fluid formerly surrounding an organ or a joint, endocrine fluid, fecal matter, semen, urine, or any of these in any combination.

23. The material of claim 20 wherein the frozen biological composition comprises a frozen culture of the biological composition.

24. The material of claim 23 wherein the frozen culture is derived from any fluid or semi-fluid biological sample, any soft tissue or hard tissues or any fragment thereof, any virus, any protozoa, any bacteria, any fungi, or any combination of any of these.

25. The material of claim 20 wherein the aldehyde has a concentration ranging from as low as about 0.1 volume percent to as high as about 0.6 volume percent, based upon the total volume of the aldehyde, alcohol, ketone, and water in the fluid composition.

26. The material of claim 20 wherein the alcohol has a concentration ranging from as low as about 25 volume percent to as high as about 45 volume percent, based upon the total volume of the aldehyde, alcohol, ketone, and water in the fluid composition.

27. The material of claim 20 wherein the ketone has a concentration ranging from as low as about 10 volume percent to as high as about 30 volume percent, based upon the total volume of the aldehyde, alcohol, ketone, and water in the fluid composition.

28. A material, the material comprising:
a biological composition; and
a fluid composition in intimate contact with the biological composition, the fluid composition comprising:
ketone;
alcohol; and
water; and
aldehyde wherein:
the aldehyde has a concentration ranging from as low as about 0.1 volume percent to as high as about 0.6 volume percent, based on the total volume of the aldehyde, alcohol, ketone, and water in the fluid composition; and
the alcohol has a concentration ranging from as low as about 25 volume percent to as high as about 45 volume percent, based upon the total volume of the aldehyde, alcohol, ketone, and water in the fluid composition.

29. The material of claim 28 wherein the aldehyde comprises formaldehyde.

30. The material of claim 28 wherein the alcohol comprises ethanol.

31. The material of claim 28 wherein the ketone comprises acetone.

32. A method, the method comprising
applying a fluid composition to a biological composition to form a fixed biological sample, the fluid composition comprising:
an aldehyde;
alcohol; and
a ketone; wherein the volumetric ratio of the alcohol to the ketone in the fluid composition ranges from as low as about 0.8:1 to as high as about 4.5:1 and the volumetric ratio of the alcohol to the aldehyde in the fluid composition ranges from as low as about 41.5:1 to as high as about 450:1; and
preparing the fixed biological sample for microscopic evaluation.

33. A method, the method comprising
applying a fluid composition to a biological composition to form a fixed biological sample, the fluid composition comprising:
an aldehyde;
alcohol; and
a ketone; wherein:
the aldehyde has a concentration ranging from as low as about 0.1 volume percent to as high as about 0.6 volume percent, based on the total volume of the aldehyde, alcohol, ketone, and water in the fluid composition; and
the volumetric ratio of the alcohol to the ketone in the fluid composition ranges from as low as about 0.8:1 to as high as about 4.5:1; and
preparing the fixed biological sample for microscopic evaluation.

34. A method, the method comprising contacting a fluid composition and a frozen biological composition, the fluid composition comprising an aldehyde, alcohol, and a ketone.

35. A method, the method comprising
providing a modified biological composition, the modified biological composition comprising a frozen processing aid; and
contacting the modified biological composition and a solvent, the frozen processing aid soluble in the solvent.
